(19) United States
(12) Patent Application Publication
(10) Pub. No.: US 2005/0042283 A1
(43) Pub. Date: Feb. 24, 2005

(54) Histamine and CCK2/gastrin receptor blockade in the treatment of acid-peptic disease and cancer

(75) Inventor: Timothy C. Wang, New York, NY (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA (US)

(21) Appl. No.: 10/889,120

(22) Filed: Jul. 12, 2004

Related U.S. Application Data
(60) Provisional application No. 60/486,667, filed on Jul. 11, 2003.

(51) Int. Cl. A61K 31/34; A61K 9/20

(52) U.S. Cl. 424/464; 514/471

(57) ABSTRACT

The present invention relates to methods useful for treating or preventing gastrointestinal disorders. More specifically, the invention relates to methods for the treatment and prevention of acid reflux disease (GERD), peptic ulcer disease, dyspepsia, gastritis, and pre-malignant and malignant disease of the stomach.

### RELATED APPLICATIONS

This patent application claims the benefit of U.S. Provisional Patent Application Ser, No. 60/486,667 entitled “Histamine and CCK2/Gastrin Receptor Blockade in the Treatment of Acid-Peptic Disease and Cancer,” filed Jul. 11, 2003. The entire content of the above-referenced provisional patent applications is incorporated herein by this reference.

### GOVERNMENT RIGHTS

This invention was made at least in part with government support under grant no. CA93405, AI37750 and RR07036 awarded by the National Institutes of Health. The government may have certain rights in this invention.

### BACKGROUND OF THE INVENTION

Gastric acid has been known for many decades to be a key factor in normal upper gastrointestinal functions, including protein digestion, calcium and iron absorption, as well as providing some protection against bacterial infections. However, inappropriate levels of gastric acid underlie several widespread pathological conditions, such as gastroesophageal reflux disease (GERD), for which heartburn is the most common symptom, and peptic ulcers, which cause pain and suffering in millions of people, and which, only thirty years ago, could be life-threatening if untreated. Treatment options are often limited. For example, the main treatment for peptic ulcers was the administration of antacids (e.g., aluminum hydroxide, magnesium hydroxide, magnesium trisilicate, calcium carbonate, and sodium bicarbonate) to neutralize excess gastric acid (which promotes ulcer formation and prevents healing). However, such course of treatment only provides temporary relief. An alternative treatment was by way of an operation (gastrectomy, in which part of the stomach is removed, and/or vagotomy, in which nerves to the stomach are sectioned). Such surgery has also lead to serious side effects. Recently, researchers have focused on achieving pharmacological control of the mechanism underlying gastric acid secretion in order to provide better treatment options for gastrointestinal disorders such as GERD and peptic ulcers. In fact, acid suppressive drugs represent one of the most commonly prescribed medications to treat symptoms of acid peptic disorders. One of the most common methods for treating acid peptic disorders involves the administration of an agent belonging to the class of histamine 2-receptor antagonists (H2RA). H2RA's act by reducing the amount of hydrochloric acid that parietal cells secrete into the lumen of the stomach. This raises the pH of the stomach contents, reducing acid-related pain thereby creating an environment where damaged tissues can heal. Some common H2RA include: cimetidine (Tagamet®), ranitidine (Zantac®), famotidine (Pepcid®), and nizatidine (Axid®). In general, H2RAs do not inhibit acid as effectively as PPIs (described below), but indeed show some efficacy in the treatment of peptic ulcer disease and GERD. The most effective agent, loxtidine, was never marketed due to the induction of ECLomas (a benign endocrine tumor of the stomach) in rats.

A new class of agents known as the proton pump inhibitors (PPIs) are among the most effective acid blockers in the treatment of acid-reflux/GERD, peptic ulcer disease and dyspepsia. PPIs function to inhibit gastric acid secretion by inhibition of the H+/K+/ATP-ase enzyme. Despite the treatment efficacy of PPIs, they do not suppress acid completely in all patients, and have the unfortunate side effect of inducing hypergastrinemia which can promote epithelial growth. In addition, the combination of PPIs and Helobacter pylori (a bacteria that infects half of the world's population) can lead to accelerated development of gastric atrophy. Atrophy is a recognized precursor of gastric cancer. Recent studies have shown that the combination of hypergastrinemia (elevated serum gastrin levels) and H. pylori infection is a potent inducer of gastric cancer in mouse models. Current PPIs used for the treatment of acid peptic disorders include: omeprazole (Prilosec®), lansoprazole (Prevacid®), pantoprazole (Protonix®), rebeprazole (Aciphex®) and esomeprazole (Nexium®).

In the last decade, a number of additional agents have been developed that antagonize the gastrin receptor (referred to interchangeably as CCK2 or CCK-B) and are referred to herein as the CCK2 receptor antagonists (CCK2RA). CCK2RAs act by blocking the gastrin receptor leading to a reduction in histamine production. CCK2RAs have not been tested in human patients; however preliminary tests show some efficacy in reducing gastric acid secretion.

Despite the varied therapies already available for the treatment of acid peptic disorders, research still continues in an attempt to find improved means of acid control.

### SUMMARY OF THE INVENTION

The present invention relates to methods useful for treating or preventing gastrointestinal diseases and disorders (e.g., peptic acid disorders). The studies exemplified herein were designed, in particular, to determine the effectiveness of combining an H2RA and a CCK2RA for the treatment of acid reflux disease (GERD), peptic ulcer disease, dyspepsia, gastritis, and pre-malignant and malignant diseases of the stomach. Accordingly, the invention provides methods of treating acid peptic diseases and disorders in a subject in need of treatment.

In one aspect, the invention features methods for treating a subject having an acid peptic disease or disorder or a proliferative disorder (e.g., gastric cancer), involving administering a therapeutically effective amount of a histamine 2-receptor antagonist (H2RA) and a gastrin receptor antagonist (e.g., CCK2RA) such that the subject is treated. Histamine 2-receptor antagonists can be reversible or irreversible inhibitors. An exemplary histamine 2-receptor antagonist is loxtidine. An exemplary gastrin receptor antagonist is YF476.

Preferably, the treatment results in a diminution in gastric acid secretion or in the reduction, inhibition, or amelioration of certain symptoms or pathogenesis of acid peptic diseases or disorders.

The invention also features pharmaceutical compositions and pharmaceutical kits for use in the claimed methodologies. Methods of co-promoting the active agents of the invention are also featured.

### BRIEF DESCRIPTION OF THE DRAWINGS

FIG. 1: Characteristics of gastrin receptor (CCK-2R or CCK-BR) antagonists. The chemical structure, the molecular weights, IC50 or Kd, and specificity (ratio of affinity for CCK-B versus CCK-A) are shown for YF476 and YM022 (Yamanouchi Pharmaceuticals Co. Ltd., Tsukuba, Japan). YF476 was tested in gastrin receptor expressing AGS-E cells transfected with HDC-luciferase promoter-reporter gene constructs. Gastrin (at 10⁻⁷ M) strongly stimulated HDC promoter activity, which was blocked completely by treatment with YF476 at 10⁻⁷ M.

FIG. 2: Characteristics of loxtidine. The chemical structure of loxtidine, and gastric histology from a South African rat (Mastery's) treated for 16 weeks with loxtidine showing enterochromaffin-like (ECL) cell neoplasia.

FIG. 3: Experimental protocol for treatment of H. felis-infected INS-GAS mice with (B) YF476 (C) loxtidine or (D) both drugs. The control group (A) included INS-GAS mice that were infected with H. felis but untreated, resulting in a total of 4 groups of mice (A-D). The INS-GAS (male) mice entered the study at 6-8 weeks of age, and were housed in 4 cages. There were 21 mice in each group, seven of which were euthanized after 3 months and 14 of which were euthanized after 6-7 months. The doses of medication are shown. Loxtidine was given in the drinking water while YF476 was given by subcutaneous injection once a week.

FIG. 4: Closed stomachs from INS-GAS/H. felis mice. Gross photos of representative stomachs from each of the four groups of 6 month H. felis-infected INS-GAS mice are shown. Mice were euthanized by CO2 inhalation and the stomachs were removed by transfection of the esophagus and duodenum. YF476 and loxtidine treatment each led to a significant reduction in apparent size, with a greater reduction seen with the combination of the two drugs.

FIG. 5. YF476 and/or loxtidine treatments for 6 months synergistically inhibited gastric tumors in H.felis-infected INS-GAS mice. (A)-(E) Outlook of stomach of H.felis-infected INS-GAS mice treated with YF476 and/or loxtidine for 6 months; (A) no drug (B) YF476 (C) loxtidine (D) YF476 and loxtidine (E) FVB control mice (F) Stomach wet-weight of YF476 and/or loxtidine treated mice (G) Body weight of YF476 and/or loxtidine treated mice (H) The ratio of stomach wet-weight over body weight of YF476 and/or loxtidine treated mice. Body weight of untreated mice were significantly smaller than YF476 and/or loxtidine-treated mice and FVB control mice, (*; p<0.01, n=6 per each group). Stomach wet-weight (F) and the ratio of stomach wet-weight over body weight (H) of YF476 and/or loxtidine-treated mice were significantly smaller than untreated mice, (*; p<0.01, n=6 per each group).

FIG. 6: YF476 and/or loxtidine treatments for 3 months synergistically inhibited gastric atrophy, hyperplasia and dysplasia in H.felis-infected INS- GAS mice. (A)-(D) Synergistic inhibition of gastric atrophy, hyperplasia and dysplasia in H.felis-infected INS-GAS mice. Representative hematoxylin and eosin stains are shown (original magnification 40×; scale bar=400 μm); (A) no drug (B) YF476 (C) loxtidine (D) YF476 and loxtidine. Treatment with either YF476 or loxtidine alone (B,C) resulted in partial inhibition of gastric atrophy and foveolar hyperplasia noted in the untreated H.felis-infected INS-GAS mice (A). In addition, the mice treated with the combination of YF476 and loxtidine (D) showed almost complete inhibition of gastric atrophy, hyperplasia and dysplasia.

FIG. 7: YF476 and/or loxtidine treatments for 3 months strongly inhibited gastric acid outputs in H.felis-infected INS-GAS mice. Gastric acid output in the four study groups of INS-GAS/H. felis mice compared to wild type (FVB/N) control mice. Acid secretion over 4 hours was measured by the pyloric ligation technique and expressed as μEq protons, (*; p<0.05 in comparison of H.felis-infected INS-GAS mice without drug treatment; n=4 per each group), as previously described (Chen D. et al.,2000) At 3 months post infection, INS-GAS/H. felis mice showed near normal gastric acid output. Loxtidine and YF476 both inhibited acid secretion by 80% and 90%, respectively, while the combination resulted in 100% suppression of acid secretion.

FIG. 8: Serum amidated gastrin levels in YF476 and/or loxtidine-treated H.felis-infected INS-GAS mice. Serum amidated gastrin levels of YF476 treated mice and YF476 plus loxtidine double treated mice for 6 months were significantly higher than untreated mice, (*; p<0.05, **; p<0.01), whereas those of loxtidine treated mice showed no significant, but in a higher tendency, when compared with untreated mice, (#:p=0.054).

FIG. 9: YF476 and/or loxtidine treatments for 6 months synergistically inhibited gastric carcinogenesis in H.felis-infected INS-GAS mice. (A)-(D) Synergistic inhibition of gastric carcinogenesis in H.felis-infected INS-GAS mice. Representative hematoxylin and eosin stains are shown, (original magnification 40×; scale bar=400 μm), (A) no drug (B) YF476 (C) loxtidine (D) YF476 and loxtidine. Treatment with either YF476 or loxtidine alone (B,C) resulted in a significant decrease in overall mucosal thickness, a partial inhibition of neoplasia and the elimination of submucosal invasion observed in untreated mice (A). In addition, treatment with the combination of YF476 and loxtidine (D) resulted in nearly complete inhibition of neoplasia and normalization of histology with only mild inflammation and edema.

FIG. 10: H. felis infection status in the stomachs of YF476 and/or loxtidine-treated H.felis-infected INS-GAS mice. (A) Warthin-Starry silver staining of H.felis in the antrum of stomach of H.felis-infected INS-GAS mice treated with YF476 plus loxtidine for 6 month detected H.felis colonies in a spiral form, (original magnification 1000×). (B) ELISA assay for mice serum IgG of H.felis-specific antibody showed no significant difference of H.felis IgG titer among YF476 and/or loxtidine treated mice versus untreated mice, (n=6 per each group). (C) Quantitative real-time PCR analysis for H.felis DNA with mice gastric corpus DNA showed no significant difference of H.felis DNA copy numbers per gastric corpus DNA (copy/μg) in YF476 alone nor loxtidine alone treated mice compared with non drug treated mice was seen, whereas a significant increase was observed in YF476 plus loxtidine double treated mice, (*;p<0.01; n=6 per each group).

FIG. 11: Growth Factor Expression Analysis in YF476 and/or Loxtidine-treated H.felis-infected INS-GAS mice. (A)-(D) Quantitative real-time PCR analysis of growth factor expression level, (A) Reg I (B) Amphiregulin (C) HB-EGF (D) TGF-alpha. All three groups of YF476 and/or loxtidine treated mice showed significantly lower level of Reg I and amphiregulin expression than non drug treated mice, whereas expression level of HB-EGF showed no significant, but in a lower tendency. TGF-alpha expression was almost unchanged among the four groups, (*; p<0.05, **; p<0.01, n=6 for each group).

FIG. 12: Loxtidine treatment of H.felis-infected INS-GAS mice showed mild shifts of cytokine expression profiles from Th1 to Th2 polarization. (A)-(C) Quantitative real-time PCR analysis of Th1 and Th2 cytokines and somatostatin expression level, (A) IFN-gamma (B) TNF-alpha (C) IL-4 (D) somatostatin, (*; p<0.05, **; p<0.01, n=6 for each group). Loxtidine alone and YF476 plus loxtidine double treated mice showed significantly lower level of INF-gamma and TNF-alpha expression compared with untreated mice, (*: p<0.05), whereas YF476 treated mice did not show a significant change. Similarly, loxtidine alone and YF476 plus loxtidine double treated mice showed significantly higher level of IL-4 and somatostatin expression compared with untreated mice, (*: p<0.05), whereas YF476 treated mice showed no significant change. (E) Serum H.felis IgGl/IgG2a Ratio, (n=6 for each group), Serum H.felis IgGl/IgG2a ratio of YF476 or loxtidine treated mice were significantly lower than untreated mice, (*;p<0.01, **; p<0.05), whereas YF476 plus loxtidine double treated mice showed no significant, but in a higher tendency, (#; p=0.054).

FIG. 13: Omeprazole treatment for 3 months resulted in the mild progression of gastric hyperplasia and dysplasia in H.felis-infected INS-GAS mice (A)-(D) Representative hematoxylin and eosin stains are shown, (original magnification; 40×, scale bar=400 μm), (A) no drug (B) omeprazole alone (C) omeprazole plus YF476 (D) omeprazole plus loxtidine. Treatment with omeprazole alone for 3 months did not show a reduction in atrophy but instead appeared to manifest a more rapid progression of gastric foveolar hyperplasia and dysplasia than untreated mice (A,B), whereas the combination of omeprazole with YF476 or loxtidine resulted in a significant suppression of gastric hyperplasia and dysplasia compared to omeprazole alone treated mice (C,D).

### DETAILED DESCRIPTION OF THE INVENTION

Acid suppressive drugs represent one of the most commonly prescribed medications for treatment of acid peptic disorders worldwide. Proton pump inhibitors (PPIs), are among the most effective acid blockers but do not suppress acid completely in all patients and also have the undesirable side effect of inducing hypergastrinemia which can promote epithelial growth. Moreover, PPIs in combination with H. pylori can lead to accelerated development of gastric atrophy, a precursor of gastric cancer. Even in the absence of H. Pylori, PPIs may cause problems including induction of achlorhydria and bacterial overgrowth which could lead to inflammation.

Histamine 2- receptor antagonists (H2RA), including cimetidine, ranitidine, famotidine and nizatidine, do not inhibit acid as effectively as PPIs but show some efficacy in treatment of peptic ulcer disease and GERD. The most effective H2RA developed in the laboratory, loxtidine, was never marketed due to its toxicity laboratory rats.

Gastrin receptor antagonists (e.g., antagonists of the CCK2 or CCK-B receptor) have shown efficacy in reducing acid secretion in animals but have not yet been tested in humans. A combination therapy featuring a PPI and a CCK2RA has been described in U.S. patent application Ser. No. 2003/0049698 A1. The idea of combining an H2RA and a CCK2RA has not heretofore been described, since based on published studies there would, in theory, be little synergy and little advantage over a PPI.

The invention is based, at least in part, on the discovery that a histamine 2 receptor antagonist (H2RA) when used in combination with a gastrin receptor antagonist (e.g., CCK2RA) leads to the reduction of gastric acid secretion, but at the same time blocks cancer development. The instant inventors have made the unexpected discovery that the combination of an H2RA and a CCK2RA has a synergistic action when used in combination to treat acid peptic disorders. In addition, the combination appears to reduce the inflammatory response to Helicobacter infection, and thus inhibit progression to atrophy and cancer of the stomach. Accordingly, the invention provides methods of treating acid peptic disorders in a subject in need of treatment. In particular, the invention features methods of treating acid reflux disease (GERD), Barrett's esophagus, peptic ulcer disease, dysplasia, gastritis (e.g., chronic gastritis), gastric atrophy and pre-malignant and malignant diseases of the upper digestive tract (stomach and esophageal cancer) (for example, gastric cancer). The combination may prove useful to treat or prevent other malignancies as well.

Accordingly, in one aspect, the invention features methods for treating a subject having an acid peptic disorder. In another aspect, the invention features methods for treating a subject having a proliferative disorder (e.g., cancer). The methods involve administering a therapeutically effective amount of a histamine 2-receptor antagonist (H2RA) and a gastrin receptor antagonist (e.g., CCK2RA) such that the subject is treated. Exemplary acid peptic disorders include, but are not limited to, acid reflux disease (GERD), peptic ulcer disease, dyspepsia, gastritis, and pre-malignant and malignant disease of the stomach. Exemplary cancers include, but are not limited to colon, ovarian, lung, breast, endometrial, uterine, hepatic, gastrointestinal, prostate, and brain cancer; tumorigenesis and metastasis; skeletal dysplasia; and hematopoietic and myeloproliferative disorders.

In one embodiment, the histamine 2-receptor antagonist is a reversible inhibitor (e.g., nizatidine, cimetideine, ranitidine, famotidine or roxatide). In another embodiment, the histamine 2-receptor antagonist is a irreversible inhibitor (e.g., loxtidine or lamitidine). An exemplary therapeutically effective amount of the histamine 2-receptor antagonist is within the range from about 0.02 to about 0.5 mg/kg/day. An exemplary therapeutically effective amount of the gastrin receptor antagonist is within the range from about 1 to 25 mg/kg/day.

Preferably, the treatment results in a diminution in gastric acid secretion. More preferably, the histamine 2-receptor antagonist and the gastrin receptor antagonist when simultaneously present in a subject act synergistically to reduce, inhibit, or ameliorate the symptoms or pathogenesis of acid peptic disorders.

In an exemplary embodiment, the histamine 2-receptor antagonist and the gastrin receptor antagonist are simultaneously administered to the subject.

The invention also features pharmaceutical compositions that include a therapeutically effective amount of a histamine 2-receptor antagonist and a gastrin receptor antagonist. Exemplary pharmaceutical compositions are formulated for oral administration (e.g., a capsule). The invention also features pharmaceutical kits where a histamine 2-receptor antagonist and a gastrin receptor antagonist are packaged in separate containers for sale or delivery to consumers. The invention also features administering a histamine 2-receptor antagonist to a subject already having a sufficient systemic level of a gastrin receptor antagonist to produce the desired synergistic therapeutic effect, e.g., treatment of an acid peptic disorder or a gastric malignancy. Alternatively, the invention features administering a gastrin receptor antagonist to a subject already having a sufficient systemic level of a histamine 2-receptor antagonist to produce the desired synergistic therapeutic effect. Promoting either a histamine 2-receptor antagonist for use in combination with a gastrin receptor antagonist (e.g., for treatment of an acid peptic disorder or a gastric malignancy), or alternatively, promoting a gastrin receptor antagonist for use with a histamine 2-receptor antagonist, is also within the scope of the instant invention.

Before further description of the invention, certain terms employed in the specification, examples and appended claims are, for convenience, collected here.

The term “subject”, as used herein, includes living organisms in which an acid peptic disorder can occur. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate gastric acid secretion in the subject as further described herein. An effective amount of the therapeutic compound necessary to achieve a therapeutic effect may vary according to factors such as the amount of H2A or CCK2RA already deposited at the clinical site in the subject, the age, sex, and weight of the subject, and the ability of the therapeutic compound to modulate gastric acid secretion in the subject. Dosage regimens can be adjusted to provide the optimum therapeutic response. For example, several divided doses may be administered daily or the dose may be proportionally reduced as indicated by the exigencies of the therapeutic situation. In an exemplary aspect of the invention, the subject is a human.

The term “gastrointestinal disorder”, as used herein, includes any disease, disorder, condition, pathology, and other abnormality relating to, affecting, or including both stomach and intestine (i.e., the gastrointestinal tract).

The term “acid peptic disorder”, as used herein, includes any disease, disorder, condition, pathology, and other abnormality associated with the secretion of gastric acid, including but not limited to peptic ulcer disease (PUD), dyspepsia, gastro-esophageal reflux disease (GERD), gastritis (e.g., chronic gastritis), gastric atrophy, pre-malignant and malignant diseases of the stomach and other disorders associated with aberrant histaminergic function (e.g., excess histamine, or insufficient histamine).

The term “histamine 2-receptor”, as used herein, refers to the cell surface receptor which binds, and signals in response, to histamine. The term “histamine 2-receptor” refers to the receptors found in (or isolated from) any species, particularly mammalian, including bovine, ovine, porcine, murine, equine, and preferably human.

The term “histamine 2-receptor antagonist” (H2RA) refers to a compound or agent that acts, for example, in cells in culture or in vivo, to reduce, decrease, diminish, or lessen a biological or physiological activity of the histamine 2-recptor elicited by histamine. Histamine 2-receptor antagonists are also referred to herein and in the art as “histamine 2-receptor blockers” or “H2 blockers”.

The term “gastrin receptor” as used herein, refers to a cell surface receptor which binds, and signals in response to, gastrin. The term “gastrin receptor” refers to a receptor found in (or isolated from) any species, particularly mammalian, including bovine, ovine, porcine, murine, equine, and preferably human.

The term “gastrin receptor antagonist”, as used herein, refers to a compound or agent that acts, for example, in cells in culture or in vivo, to reduce, decrease, diminish, or lessen a biological or physiological activity of a gastrin receptor elicited by gastrin. Preferably, “gastrin receptor antagonists” bind to the CCK-B/gastrin receptor and inhibit secretion of gastric acid via the CCK-B/gastrin receptor. Alternatively, a “gastrin receptor antagonist” can bind to a non-CCK-B/gastirn receptor, for example, another CCK receptor family member that binds gastrin.

The terms “antagonist” or “inhibitor” as used herein, refers to a molecule which, when interacting with a biologically active molecule, blocks or modulates the biological activity of the biologically active molecule. Antagonists and inhibitors include, but are not limited to, proteins, nucleic acids, carbohydrates, lipids or any other molecules that bind or interact with biologically active molecules. Antagonists and inhibitors can effect the biology of entire cells, organs, or organisms (e.g., an inhibitor that slows or prevents the secretion of gastic acid).

The term “reversible inhibitor” or “reversible antagonist”, as used herein, refers to an inhibitor or antagonist capable of readily dissociating from the biologically active molecule with which it associates (e.g., a receptor), thereby forming a short-lasting or transient combination with the biologically active molecule (e.g., receptor). The term “reversible inhibitor” is used interchangeably herein with the term “competitive inhibitor”. A “reversible histamine 2-receptor agonist” or “inhibitor” is defined as a competitive inhibitor of the action of histamine at the histamine receptors, including receptors on the gastric cells. Preferred reversible histamine 2-receptor agonists or inhibitors include, but are not limited to, nizatidine (Axid™), cimetidine (Tagamet™), ranitidine (Zantac™), famotidine (Pepcid™) and roxatidine.

The term “irreversible inhibitor” or “reversible antagonist”, as used herein, refers to an inhibitor or agonist which forms a stable chemical bond with the biologically active molecule with which it associates (e.g., a receptor), thereby forming a long-lasting combination with the biologically active molecule (e.g., receptor). The term “irreversible inhibitor” is used interchangeably herein with the term “non-competitive inhibitor”. An “irreversible histamine 2-inhibitor” or “antagonist” is defined as a non-competitive inhibitor of the action of histamine at the histamine receptor, including receptors on the gastric cells. Preferred irreversible histamine 2-receptor agonists or inhibitors include, but are not limited to, loxtidine and lamitidine.

The term “treatment”, as used herein, is defined as the application or administration of a therapeutic agent to a patient, or application or administration of a therapeutic agent to an isolated tissue or cell line from a patient, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, small molecules, peptides, antibodies, ribozymes and antisense oligonucleotides.

The term “effective amount”, as used here in, is defined as that amount necessary or sufficient to treat or prevent a gastrointestinal disorder (e.g., an acid peptic disorder), e.g., to prevent the various symptoms of the disorder. The effective amount can vary depending on such factors as the size and weight of the subject, the type of illness, or the particular anti-gastric agent. For example, the choice of the anti-gastric agent can affect what constitutes an “effective amount.” One of ordinary skill in the art would be able to study the aforementioned factors and make the determination regarding the effective amount of the anti-gastric agent without undue experimentation.

In another aspect, the invention relates to a method where at least the first compound is for preventing, reducing, or inhibiting gastric acid production in a subject. For example, such a method comprises administering to a subject a therapeutically effective amount of a pharmaceutical composition capable of inhibiting an acid peptic disorder

The term, a “cellular growth or proliferation disorder”, as used herein includes a disease or disorder that affects a cell growth or proliferation process. As used herein, a “cellular growth or proliferation process” is a process by which a cell increases in number, size or content, by which a cell develops a specialized set of characteristics which differ from that of other cells, or by which a cell moves closer to or further from a particular location or stimulus. A cellular growth or proliferation process includes the metabolic processes of the cell and cellular transcriptional activation mechanisms. A cellular growth or proliferation disorder may be characterized by aberrantly regulated cell growth, proliferation, differentiation, or migration. Cellular growth or proliferation disorders include tumorigenic disease or disorders. As used herein, a “tumorigenic disease or disorder” includes a disease or disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of cellular growth or proliferation disorders include, but are not limited to, cancer, e.g., carcinoma, sarcoma, or leukemia, examples of which include, but are not limited to, colon, ovarian, lung, breast, endometrial, uterine, hepatic, gastrointestinal, prostate, and brain cancer; tumorigenesis and metastasis; skeletal dysplasia; and hematopoietic and/or myeloproliferative disorders.

The term “pharmaceutical composition” as used herein, means one or more compatible solid or liquid filler diluents or encapsulating substances which are suitable for administration to a human or lower animal.

I. Histamine H2 Receptor and Histamine H2 Receptor Antagonists

Histamine is a biogenic amine, i.e., an amino acid that possesses biological activity mediated by pharmacological receptors after decarboxylation. The role of histamine in immediate type hypersensitivity is well established. (Plaut, M. and Lichtenstein, L. M. 1982 Histamine and immune responses. In Pharmacology of Histamine Receptors, Ganellin, C. R. and M. E. Parsons eds. John Wright & Sons, Bristol pp. 392-435.) Histamine produces its pathological effects by binding to a receptor located on the membrane of cells in many tissues.

The receptors for histamine, which are part of a superfamily known as the G-protein coupled receptors (GPCRs), are seven transmembrane proteins. Histamine receptors are further divided into subtypes, known as H1, H2 and H3. The type of histamine receptor expressed on cells is tissue specific. Thus, Hi is found in smooth muscles of intestine, uterus, bronchi, urinary bladder, fine blood vessels and brain. H2 is expressed in stomach, smooth muscles of airway, and blood vessels of heart, and immunoreactive cells. H3 is expressed in brain and lung. The pathological effects of histamine in hypersensitivity reactions appear primarily due to the interaction of histamine with the H1 receptor. Histamine's activity is mediated by several different subtypes of the histamine receptors. The histamine receptor 1 (H1R) is involved in vascular dilation and smooth muscle contraction. Receptor subtype 2 (H2R) is found at high levels in the stomach, but in lower numbers in the heart, brain, smooth muscle, and cells of the immune system. In the stomach, it is present in gastric parietal cells where stimulation of it leads to gastric acid secretion.

The discovery of selective antagonists for the H2 receptor revolutionized the treatment of gastric ulcers, providing specific drugs which could target the gastric mucosa, without affecting other histaminergic processes. For a review, see, e.g., Del Valle and Gantz, Am. J. Physiol., 236:G987-G996, 1997. In addition to its well known role in gastric acid secretion, the H2 receptor is also involved in other processes, including, e.g., gastrointestinal motility, intestinal secretion, cell growth, and differentiation. Histamine is the “final common mediator” of acid secretion and binds to the histamine-2 (H2) receptor on the parietal cell to stimulate HCL (acid) secretion. Histamine is produced by the enzyme histidine decarboxylase in ECL cells in the stomach, and the production of histamine is largely controlled by the circulating hormone gastrin.

The following teachings relate to classes of chemical antagonists, i.e., small molecule antagonists of the histamine 2-receptor. However, it will be appreciated by the skilled artisan that any antagonist of the histamine 2-receptor, including but not limited to, proteins, nucleic acids, carbohydrates, lipids or any other molecules that bind or interact with the histamine 2-receptor can be utilized in the combination therapies described herein.

Moreover, indirect means of antagonizing histamine 2-receptors are known in the art and can be used in the combination therapies described herein. Finally, additional means of blocking histamine production and/or release which do not act via histamine 2-receptors are known in the art. The skilled artisan will appreciate that such means could be utilized, in combination with gastrin receptor antagonism, to facilitate the therapeutic treatments of the instant invention. The term “histamine blocker” or “histamine inhibitor” can be substituted for the term “H2RA” throughout the claims and detailed description set forth herein to provide detailed description of this aspect of the invention. Preferred are compounds of agents that inhibit histamine production, i.e., “histamine inhibitors”. An exemplary class of histamine inhibitors are compounds/agents that inhibit the enzyme histidine decarboxylase (HDC). A preferred HDC inhibitor is alpha-fluromethylhistidine (α-FMH), an irreversible inhibitor of HDC.

The subject invention involves the use of compounds which specifically blockade the receptors involved in mepyramine-insensitive, non-H-1 (H-2), histamine responses, and which do not blockade the receptors involved in mepyramine-sensitive histamine responses.

Selective H-2 antagonists are those compounds found to be H-2 antagonists through their performance in classical preclinical screening tests for H-2 antagonist function. Selective H-2 antagonists are identified as compounds which can be demonstrated to function as competitive or non-competitive inhibitors of histamine-mediated effects in those screening models specifically dependent upon H-2 receptor function, but to lack significant histamine antagonist activity in those screening models dependent upon H-1 receptor function. Specifically, this includes compounds that would be classified as described by Black, J. W., W. A. M. Duncan, C. J. Durant, C. R. Ganellin & E. M. Parsons, “Definition and Antagonism of Histamine H₂ -Receptors”, Nature, Vol. 236 (Apr. 21, 1972), pp. 385-390 (Black), incorporated herein by reference, as H-2 antagonists if assessed as described by Black through testing with the guinea pig spontaneously beating right atria in vitro assay and the rat gastric acid secretion in vivo assay, but shown to lack in significant H-1 antagonist activity relative to H-2 antagonist activity, if assessed as described by Black with either the guinea pig ileum contraction in vitro assay or the rat stomach muscle contraction in vivo assay. Preferably selective H-2 antagonists demonstrate no significant H-1 activity at reasonable dosage levels in the above H-1 assays. (A typical reasonable dosage level is the lowest dosage level at which 90% inhibition of histamine, preferably 99% inhibition of histamine, is achieved in the above H-2 assays).

Selective H-2 antagonists include compounds which are disclosed in US Pat. Nos. 5,294,433 and 5,364,616 Singer et al., issued 15 Mar. 1994 and 15 Nov. 1994 respectively and assigned to Procter & Gamble, wherein the selective H-2 antagonist is selected from the group consisting of cimetidine, etintidine, ranitidine, ICIA-5165, tiotidine, ORF-17578, lupitidine, donetidine, famotidine, roxatidine, pifatidine, lamtidine, BL-6548, BMY-25271, zaltidine, nizatidine, mifentidine, BMY-52368, SYF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728 and HB-408. Particularly preferred is loxtidine, 1 -methyl-5-((3-(3-(1 -piperidinylmethyl)phenoxy)propyl)amino)-1H-1,2,4-triazole-3-ethanol. The combination therapies of the instant invention are exemplified herein using loxtidine. Loxtidine is proposed to inhibit, for example, inflammation caused by induction of achlorhydria and/or bacterial overgrowth (undesirable side effects of using PPI therapy alone.

Selective H-2 antagonists include compounds meeting the above criteria which are disclosed in the following U.S. Pat. Nos.: 3,751;470; 3,876,647; 3,881,016; 3,891,764; 3,894,151; 3,897,444; 3,905,964; 3,910,896; 3,920,822; 3,932,443; 3,932,644; 3,950,333; 3,968,227; 3,971,786; 3,975,530; 3,979,398; 4,000,296; 4,005,205; 4,024,271; 4,034,101; 4,035,374; 4,036,971; 4,038,408; 4,056,620; 4,056,621; 4,060,621; 4,062,863; 4,062,967; 4,070,472; 4,072,748; 4,083,983; 4,083,988; 4,084,001; 4,090,026; 4,093,729; 4,098,898; 4,104,381; 4,104,472; 4,105,770; 4,107,319; 4,109,003; 4,112,104; 4,112,234; Re. 29,761; 4,118,496; 4,118,502; 4,120,966; 4,120,968; 4,120,972; 4,120,973; 4,128,658; 4,129,657; 4,133,886; 4,137,319; 4,139,624; 4,140,783; 4,145,546; 4,151,289; 4,152,443; 4,152,453; 4,153,793; 4,154,834; 4,154,838; 4,156,727; 4,157,347; 4,158,013; 4,160,030; 4,165,377; 4,165,378; 4,166,856; 4,166,857; 4,169,855; 4,170,652; 4,173,644; 4,181,730; 4,185,103; 4,189,488; 4,190,664; 4,191,769; 4,192,879; 4,197,305; 4,200,578; 4,200,760; 4,203,909; 4,210,652; 4,210,658; 4,212,875; 4,215,125; 4,215,126; 4,216,318; 4,218,452; 4,218.466; 4,219,553; 4,220,767; 4,221,737; 4,227,000; 4,233,302; 4,234,588; 4,234,735; 4,238,493; 4,238,494; 4,239,769; Re. 30,457; 4,242,350; 4,242,351;.4,247,558; 4,250,316; 4,252,819; 4,255,425; 4,255,440; 4,260,744; 4,262,126; 4,264,608; 4,264,614; 4,265,896; 4,269,844; 4,271,169; 4,276,297; 4,276,301; 4,279,819; 4,279,911; 4,282,213; 4,282,221; 4,282,224; 4,282,234; 4,282,363; 4,283,408; 4,285,952; 4,288,443; 4,289,876; 4,293,557; 4,301,165; 4,302,464; 4,304,780; 4,307,104; 4,308,275; 4,309,433; 4,309,435; 4,310,532; 4,315,009; 4,317,819; 4,318,858; 4,318,913; 4,323,566; 4,324,789; 4,331,668; 4,332,949; 4,333,946; 4,336,394; 4,338,328; 4,338,447; 4,338,448; 4,341,787; 4,342,765; 4,347,250; 4,347,370; 4,359,466; 4,362,728; 4,366,164; 4,372,963; 4,374,248; 4,374,251; 4,374,839; 4,374,843; 4,375,435; 4,375,472; 4,375,547; 4,379,158; 4,380,638; 4,380,639; 4,382,090; 4,382,929; 4,383,115; 4,385,058; 4,386,099; 4,386,211; 4,388,317; 4,388,319; 4,390,701; 4,394,508; 4,395,419; 4,395,553; 4,399,142; 4,405,621; 4,405,624; 4,407,808; 4,410,523; 4,413,130; 4,426,526; 4,427,685; 4,432,983; 4,433,154; 4,435,396; 4,438,127; 4,439,437; 4,439,444; 4,439,609; 4,440,775; 4,442,110; 4,443,613; 4,447,441; 4,447,611; Re. 31,588; 4,450,161; 4,450,168; 4,451,463; 4,452,985; 4,452,987; 4,458,077; 4,461,900; 4,461,901; 4,464,374; 4,465,841; 4,466,970; 4,467,087; 4,468,399; 4,470,985; 4,471,122; 4,474,790; 4,474,794; 4,476,126; 4,481,199; 4,482,552; 4,482,563; 4,482,566; 4,485,104; 4,490,527; 4,491,586; 4,492,711; 4,493,840; 4,496,564; 4,496,571; 4,499,101; 4,500,462; 4,501,747; 4,503,051; 4,507,296; 4,507,485; 4,510,309; 4,510,313; 4,514,408; 4,514,413; 4,515,806; 4,518,598; 4,520,025; 4,521,418; 4,521,625; 4,522,943; 4,523,015; 4,524,071; 4,525,477; 4,526,973; 4,526,995; 4,528,375; 4,528,377; 4,528,378; 4,529,723; 4,529,731; 4,536,508; 4,537,779; 4,537,968; 4,539,207; 4,539,316; 4,540,699; 4,543,352; 4,546,188; 4,547,512; 4,548,944; 4,550,118; 4,551,466; 4,558,044; 4,558,128; 4,559,344; 4,560,690; 4,564,623; 4,567,176; 4,567,191; 4,570,000; 4,571,394; 4,571,398; 4,574,126; 4,578,388; 4,578,459; 4,578,471; 4,584,384; 4,585,781; 4,587,254; 4,588,719; 4,588,826; 4,590,192; 4,590,299; 4,595,683; 4,595,758; 4,596,811; 4,599,346; 4,600,720; 4,600,779; 4,600,780; 4,604,465; 4,607,105; 4,607,107; 4,608,380; 4,612,309; 4,613,596; 4,613,602; 4,621,142; 4,622,316; 4,632,927; 4,632,993; 4,634,701; 4,638,001; 4,639,442; 4,639,523; 4,643,993; 4,644,006; 4,645,841; 4,647,559; 4,649,141; 4,649,145; 4,649,150; 4,650,893; 4,652,572; 4,652,575; 4,656,176; 4,656,180; 4,657,908; 4,659,721; 4,663,331; 4,665,073; 4,666,932; 4,668,673; 4,668,786; 4,670,448; 4,673,747; 4,675,406; 4,681,883; 4,683,228; 4,687,856; 4,692,445; 4,692,456; 4,692,531; 4,694,008; 4,696,933; 4,699,906; 4,699,915; 4,704,388; 4,705,873; 4,710,498; 4,716,228; 4,722,925; 4,727,081; 4,727,169; 4,728,655; 4,732,980; 4,738,960; 4,738,969; 4,738,983; 4,742,055; 4,743,600; 4,743,692; 4,745,110; 4,746,672; 4,748,164; 4,748,165; 4,749,790; 4,758,576; 4,760,075; 4,762,932; 4,764,612; 4,767,769; 4,769,473; 4,772,704; 4,777,168; 4,777,179; 4,788,184; 4,788,187; 4,788,195; 4,795,755; 4,806,548; 4,808,569; 4,814,341; 4,816,583; 4,837,316; 4,847,264; 4,851,410; 4,871,765; 4,886,910; 4,886,912; 4,894,372; 4,904,792; 4,912,101; 4,912,132; 4,937,253; 4,952,589; 4,952,591; 4,957,932; 4,965,365; 4,972,267; 4,988,828; 5,008,256; 5,021,429; 5,025,014; 5,037,837; 5,037,840; 5,047,411.

Selective H-2 antagonists include compounds meeting the above criteria which are disclosed in the following European Patent Applications: 7,326; 10,893; 17,679; 17,680; 29,303; 31,388; 32,143; 32,916; 49,049; 50,407; 57,227; 67,436; 73,971; 74,229; 79,297; 80,739; 86,647; 89,765; 103,503; 103,390; 104,611; 105,703; 112,637; 122,978; 134,096; 141,119; 141,560; 156,286; 169,969; 171,342; 172,968; 173,377; 178,503; 180,500; 181,471; 186,275; 204,148; 213,571; 277,900; 355,612; 417,751; 445,949; 454,449; 454,469.

Selective H-2 antagonists include compounds meeting the above criteria which are disclosed in World Patent Application No. 91-10,656. Selective H-2 antagonists include compounds meeting the above criteria which are disclosed in the following U.K. Patent Applications: 1,341,590; 1,531,237; 1,565,647; 1,574,214; 2,001,624; 2,067,987; 2,094,300; 2,117,769; 2,124,622; 2,146,331; 2,149,406; 2,162,174; 2,209,163.

Selective H-2 antagonists include compounds meeting the above criteria which are disclosed in the following Belgian Patent Applications: 857,218; 857,219; 866,155; 884,820; 892,350; 905,235; 1,000,307.

Selective H-2 antagonists include compounds meeting the above criteria which are disclosed in the following German Patent Applications: 3,044,566; 3,341,750; 3,644,246.

Selective H-2 antagonists include compounds meeting the above criteria which are disclosed in the following French Patent Applications: 2,515,181; 2,531,703.

Selective H-2 antagonists include compounds meeting the above criteria which are disclosed in the following Spanish Patent Applications: 85-06,610; 86-05,244.

Selective H-2 antagonists include compounds meeting the above criteria which are disclosed in Netherlands Patent Application No. 88-02,089.

Selective H-2 antagonists include compounds meeting the above criteria which are disclosed in South African Patent Application No. 83-05,356.

Selective H-2 antagonists include compounds meeting the above criteria which are disclosed in the following Japanese Patent Applications: 53/005,180; 54/106,468; 55/053,247; 55/115,860; 55/115,877; 56/135,479; 57/054,177; 57/165,348; 57/169,452; 58/015,944; 58/072,572; 58/072,573; 58/090,569; 59/007,172; 59/010,582; 59/093,050; 59/093,051; 59/190,973; 60/197,663; 60/226,180; 60/228,465; 60/237,082; 61/063,665; 61/063,676; 61/115,072; 62/005,969; 62/126,169; 63/122,679; 63/183,563; 02/000,178; 02/056,449; 03/251,571.

Selective H-2 antagonists include the substituted thioalkyl-, aminoalkyl-and oxyalkyl-guanidines meeting the above criteria which are disclosed in U.S. Pat. No. 3,950,333 issued to Durant, Emmett & Ganellin on Apr. 13, 1976. Particularly preferred is cimetidine (SKF-92334), N-cyano-N′-methyl-N″-(2-(((5-methyl-1H-imidazol-4-yl)methyl)thio)ethyl)guanidine.

Cimetidine is also disclosed in the Merck Index, 11 th edition (1989), p. 354 (entry no. 2279), and Physicians' Desk Reference, 46th edition (1992), p. 2228. Related preferred H-2 antagonists include burimamide and metiamide.

Selective H-2 antagonists include the imadazolylmethylthioethyl alkynyl guanidines meeting the above criteria which are disclosed in U.S. Pat. No. 4,112,234 issued to Crenshaw & Luke on Sep. 5, 1978. Preferred is etintidine (BL-5641, BL-5641A), N-cyano-N′-(2-(((5-methyl-1H-imidazol-4-yl)methyl)thio)ethyl)-N″-2-propynyl-guanidine.

Selective H-2 antagonists include the aminoalkyl furan derivatives meeting the above criteria which are disclosed in U.S. Pat. No. 4,128,658 issued to Price, Clitherow & Bradshaw on Dec. 5, 1978. Particularly preferred is ranitidine, especially its hydrochloride salt (AH-19065). Ranitidine is N-(2-(((5-((dimethylamino)methyl)-2-furanyl)methyl)thio)ethyl)-N′-methyl-2-nitro-1,1-ethenediamine.

Ranitidine is also disclosed in the Merck Index, 11 th edition (1989), p. 1291 (entry no. 8126), and Physicians' Desk Reference, 46th edition (1992), p. 1063. Related preferred compounds include hydroxymethyl ranitidine; ranitidine bismuth citrate (GR-122311, GR-122311X); and AH-18801, N-cyano-N′-(2-(((5-((dimethylamino)methyl)-2-furanyl)methyl)thio)ethyl)-N″-methyl-guanidine.

Selective H-2 antagonists include the guanidine derivatives of imidazoles and thiazoles meeting the above criteria which are disclosed in U.S. Pat. No. 4,165,377 issued to Jones and Yellin on Aug. 21, 1979. Preferred is ICIA-5165, N-(4-(2-((aminoiminomethyl)amino)-4-thiazolyl)butyl)-N′-cyano-N″-methyl-guanidine.

Selective H-2 antagonists include the guanidine derivatives of imidazoles and thiazoles meeting the above criteria which are disclosed in U.S. Pat. No. 4,165,378 issued to Gilman, Wardleworth, and Yellin on Aug. 21, 1979. Preferred is tiotidine (ICI-125211). 125211), N-(2-(((2-((aminoiminomethyl)amino)A-thiazolyl)methyl)thio)ethyl)-N′-cyano-N″-methylguanidine.

Selective H-2 antagonists ethylene compounds meeting the above criteria which are disclosed in U.S. Pat. No. 4,203,909 issued to Algieri & Crenshaw on May 20, 1980. Preferred is ORF-17578, N-(2-(((5-((dimethylamino)methyl)-2-furanyl)methyl)thio)ethyl)-2-nitro-N′-2-propynyl,1-ethene diamine.

Selective H-2 antagonists include the substituted pyrimidine compounds meeting the above criteria which are disclosed in U.S. Pat. No. 4,234,588 issued to Brown & Ife on Nov. 18, 1980. Preferred are lupitidine (SKF-93479), 2-((2-(((5-((dimethylamino)methyl)-2-uranyl)methyl)thio)amino) -5-((6-methyl-3-pyridinyl)methyl) -4(1H)-pyrimidinone; and donetidine (SKF-3574), 5-((1,2-dihydro-2-oxo-4-pyridinyl)methyl) -2-((2-(((5-(dimethylamino)methyl)-2-furanyl)methyl)thio)ethyl)amino) -4(1H)-pyrimidinone. Also preferred are related compounds SKF-93828, 2-((2-(5-((4-(dimethylaminomethyl)-2-pyridyl)methyl)thio)ethyl)amino) -5-(2-methyl-5-pyridyl)pyrimidin-4-one; and SKF-93996, the 2-(4-(4-(dimethylaminomethyl)-2-pyridyl)butylamino) analogue of SKF 93828.

Selective H-2 antagonists include the 3-amino-5-(4-pyridyl)-1,2,4-triazole derivatives meeting the above criteria which are disclosed in U.S. Pat. No. 4,276,297 issued to Lipinski on Jun. 30, 1981. Preferred is 3-amino-5-(2-(ethylamino)-4-pyridyl)-1,2,4-triazole include the guanidine derivatives of imidazoles and thiazoles meeting the above criteria which are disclosed in U.S. Pat. No. 4,165,377 issued to Jones & Yellin on Aug. 21, 1979. Preferred is ICIA-5165, N-(4-(2-((aminoiminomethyl)amino)-4-thiazolyl)butyl) -N′-cyano-N″-methyl-guanidine.

Selective H-2 antagonists include the guanidinothazole compounds meeting the above criteria which are disclosed in U.S. Pat. No. 4,283,408 issued to Hirata, Yanagisawa, Ishii, Tsukamoto, Ito, Isomura & Takeda on Aug. 11, 1981. Preferred is famotidine (YM- 11170, MK-208), 3-(((2-((aminoiminomethyl)amino)-4-thiazolyl)methyl)thio) -N-aminosulfonyl) propanimidamide.

Famotidine is also disclosed in the Merck Index, 11th edition (1989), p. 617 (entry no. 3881), and Physicians' Desk Reference, 46th edition (1992), p. 1524.

Selective H-2 antagonists include the phenoxypropylamine derivatives meeting the above criteria which are disclosed in U.S. Pat. No. 4,293,557 issued to Shibata, Itaya, Yamakoshi, Kurata, Koizumi, Tarutani, Sakuma & Konishi on Oct. 6, 1981. Preferred is roxatidine (Hoe-062, TZU-9368), 2-hydroxy-N-(3-(3-(1-piperidinylmethyl)phenoxy)propyl) -acetamide; and roxatidine acetate (pifatidine, Hoe-760, TZU-0460), 2-(acetyloxy)-N-(3-(3-(1-piperidinylmethyl)phenoxy)propyl)-acetamide: Roxatidine acetate is also disclosed in the Merck Index, 11 th edition (1989), p. 1316 (entry no. 8252).

Selective H-2 antagonists include the 1,2,4-triazole-3,5-diamine derivatives meeting the above criteria which are disclosed in U.S. Pat. No. 4,318,913 issued to Clitherow, Bradshaw, Mackinnon, Price, Martin-Smith & Judd on Mar. 9, 1982, Preferred is lamtidine (AH-22216), 1-methyl-N5-(3-(3-1-piperidinylmethyl)phenoxy)propyl) -1H-1,2,4-triazole-3,5-diamine. Also preferred are related compounds AH-21201 and AH-21272.

Selective H-2 antagonists include the 2-guanidino-4-heteroarylthiazoles meeting the above criteria which are disclosed in U.S. Pat. No. 4,374,843 issued to LaMattina & Lipinski on Feb. 22, 1983. Preferred is zaltidine (CP-57361-01), (4-(2-methyl-1H-imidazol-4-yl) -2-thiazolyl)-guanidine.

Selective H-2 antagonists include the N-alkyl-N′-((2-(aminoalkyl)4-thiazolymethyl)thioalkyl)guanidines, thioureas, ethenediamines and related compounds meeting the above criteria which are disclosed in U.S. Pat. No. 4,375,547 issued to Ploch on Mar. 1, 1983. Preferred is nizatidine (LY-139037, ZL-101), N-(2-(((2-((dimethylamino)methyl) -4-thiazolyl)methyl)thio)ethyl)-N′-methyl-2-nitro- 1, 1-ethenediamine.

Nizatidine is also disclosed in the Merck Index, 11th edition (1989), p. 1052 (entry no. 6582), and Physicians' Desk Reference, 46th edition (1992), p. 1246.

Selective H-2 antagonists include the imidazolylphenyl amidines meeting the above criteria which are disclosed in U.S. Pat. No. 4,386,099 issued to Cereda, Donetti, Soldato & Bergamaschi on May 31, 1983. Preferred is mifentidine (DA-4577), N-(4-(1H-inidazol-4-yl) phenyl)-N′-(1-methylethyl)methanimidamide.

Mifentidine and its dihydrochloride salt are disclosed in the Merck Index, 11th edition (1989), p. 973 (entry no. 6108).

Selective H-2 antagonists include the 1-(substituted amino)-2-(amino or substituted amino)cyclobutene-3,4-diones meeting the above criteria which are disclosed in U.S. Pat. No. 4,390,701 issued to Algieri & Crenshaw on Jun. 28, 1983. Preferred are BMY-25368 (SKF-94482), 3-amino-4-((3-(3-(1-piperidinylmethyl)phenoxy)propyl)amino) -3-cyclobutene-1,2-dione and its hydrochloride salt.

Selective H-2 antagonists include the 3-(hydroxy or amino)-4-(substituted amino)- and 3,4-di(substituted amino)- 1,2,5-thiadiazole 1-oxides and 1,1-dioxides meeting the above criteria which are disclosed in U.S. Pat. No. 4,394,508 issued to Crenshaw & Aigiere on Jul. 19, 1983. Preferred is BL-6341A (BMY-26539), (4-(((2-((4-amino-1,2,5-thiadiazol-3-yl) amino)ethyl)thio)methyl)-2-thiazolyl)-guanidine, S-oxide.

Selective H-2 antagonists include the cycloalkylamino derivatives meeting the above criteria which are disclosed in U.S. Pat. No. 4,427,685 issued to Stemp on Jan. 24, 1984. Preferred is N-(2-(((5-dimethylaminomethyl-2-furanyl)methyl)thio)ethyl)-N′-cyclo-octyl-2-nitro-1,1′-ethenediamine.

Selective H-2 antagonists include alcohol guanidine derivatives meeting the above criteria which are disclosed in U.S. Pat. No. 4,451,463 issued to Large on May 29. 1984. Preferred is ICI-162846, 3-((imino((2,2,2-trifluoroethyl)amino)methyl)amino) -1H-pyrazole-1-pentanamide.

Selective H-2 antagonists include the thioalkylamide of nicotinic add 1-oxide compounds meeting the above criteria which are disclosed in U.S. Pat. No. 4,474,790 issued to Nisato & Boveri on Oct. 2, 1984. Preferred is ramixotidine (CM-57755), N-(2-(((5-((dimethylamino)methyl) -2-furanyl)methyl)thio)ethyl)-3-pyridinecarboxamide 1-oxide.

Selective H-2 antagonists include the benzo-fused heterocyclic compounds meeting the above criteria which are disclosed in U.S. Pat. No. 4,490,527 issued to Schiehser & Strike on Dec. 25, 1984. Preferred is Wy-45727, N-(2-(((5-dimethylamino)methyl) -2-furanyl)methyl)thio)ethyl)thieno(3,4-d)isothiazol-3-amine 1,1 -dioxide.

Selective H-2 antagonists include the N-substituted nicotin amide 1-oxide compounds meeting the above criteria which are disclosed in U.S. Pat. No. 4,514,408 issued to Nisato & Boveri on Apr. 30, 1985. Preferred is SR-58042, (N-(3-(3-(3-methyl)piperidinomethyl)phenoxy)propyl) -3-pyridinecarboxamide 1-oxide.

Selective H-2 antagonists include the 3-(amino or substituted amino)-4-(substituted amino) -1,2,5-thiadiazoles meeting the above criteria which are disclosed in U.S. Pat. Nos. 4,528,377 and 4,600,779 issued to Crenshaw & Algieri on Jul. 9, 1985 and Jul. 15, 1986, respectively. Preferred is BMY-25405, N-(3-(3-(1-piperidinylmethyl)phenoxy)propyl) -1,2,5-thiadiazole-3,4-diamine monohydrochloride.

Selective H-2 antagonists include the triazole amine derivatives meeting the above criteria which are disclosed in U.S. Pat. No. 4,536,508 issued to Clitherow, Price, Bradshaw, Martin-Smith, Mackinnon, Judd & Hayes on Aug. 20, 1985. Preferred is loxtidine (AH-23844), 1-methyl-5-((3-(3-(1-piperidinylmethyl)phenoxy)propyl)amino)-1H-1,2,4-triazole-3-ethanol.

Selective H-2 antagonists include the guanidino-heterocyclyl-phenylamidines meeting the above criteria which are disclosed in U.S. Pat. Nos. 4,548,944 and 4,645,841 issued to Bietti, Cereda, Donetti, Soldato, Giachetti & Micheletti on Oct. 22, 1985, and Feb. 24, 1987, respectively. Preferred is DA-4634, (4-(3-(((methylamino)methylene)amino)phenyl) -2-thiazolyl)-guanidine.

Selective H-2 antagonists include the amidine derivatives of 2-substituted 4-phenylimidazole compounds meeting the above criteria which are disclosed in U.S. Pat. No. 4,649,150 issued to Bietti, Cereda, Donetti, Giachetti & Pagani on Mar. 10, 1987. Preferred is bisfentidine (DA-5047), N-(1-methylethyl)-N′-(4-(2-methyl-1H-imidazol-4-yl)phenyl)-ethanimidamide.

Selective H-2 antagonists include the triazole amine compounds meeting the above criteria which are disclosed in U.S. Pat. No. 4,670,448 issued to Clitherow, Bradshaw, MacKinnon, Judd, Bays, Hayes & Pearce on Jun. 2, 1987. Preferred is sufotidine (AH-25352), 1-methyl-3-((methylsulfonyl)methyl)-N-(3-(3-(1-piperidinylmethyl)phenoxy)propyl) -1H-1,2,4-triazol-5-amine.

Selective H-2 antagonists include the sulfonamidines meeting the above criteria which are disclosed in U.S. Pat. No. 4,728,755 issued to Foguet, Anglada, Castello, Sacristan & Ortiz on Mar. 1, 1988. Preferred is ebrotidine (FI-3542), N-(((2-(((2-((aminoiminomethyl)amino) -4-hiazolyl)methyl)thio)ethyl)amino)methylene)-4-bromo-benzenesulfonamide.

Selective H-2 antagonists include the 1,3,4-thiadiazole derivatives meeting the above criteria which are disclosed in U.S. Pat. No. 4,738,960 issued to Schickaneder, Heter, Wegner, Schunack, Szelenyi, Postius & Ahrens on Apr. 19, 1988. Preferred is HE-30-256, 1-(3-(3-(piperidinomethyl)phenoxy)propylamino)-5-pyridin-2-sulfenamido-1,3,4-thiadiazole.

Selective H-2 antagonists include the ethylenediamine and guanidine-derivatives meeting the above criteria which are disclosed in U.S. Pat. No. 4,738,983 issued to Emig, Scheffier, Thiemer & Weischer on Apr. 19, 1988. Preferred is D-16637, N-(2(((5-((tricyclo(2,2,1,0) hept-3-ylamino)methyl-2-furanyl)methyl)thio)ethyl)-N-methyl-2-nitro-1,1-ethenediamine HCl.

Selective H-2 antagonists include the 4-aminomethyl-pyridyl-2-oxy derivatives meeting the above criteria which are disclosed in U.S. Pat. Nos. 4,912,101 and 4,977,267 issued to Hirakawa, Kashiwaba, Matsumoto, Hosoda, Sekine, Isowa, Yamaura, Sekine & Nishikawa on Mar. 27 and Dec. 11, 1990, respectively. Preferred is FRG-8813, N-(4-(4-(piperidinomethyl)pyridyl-2-oxy)-(Z)-2-butenyl)-2-(furfurylsulfinyl)acetamide.

Selective H-2 antagonists include the alkylamide derivatives meeting the above criteria which are disclosed in U.S. Pat. No. 4,837,316, issued to Sekine, Hirakawa, Kashiwaba, Yamaura, Harada, Katsuma, Matsumoto, Sekine & Isowa on Jun. 6, 1989. Preferred is FRG-8701, N-(3-(3-(piperidinomethyl)phenoxy)propyl)-2-(furfurylsulfinyl)acetamide.

Selective H-2 antagonists include the N,N′-disubstituted guanidine compounds meeting the above criteria which are disclosed in U.K. Patent Specification No. 1,531,237 of Durant, Ganellin & Parsons published on Nov. 8, 1978. Preferred is impromidine.

Selective H-2 antagonists include the 3,4-diamino-1,2,5-thiadiazole compounds meeting the above criteria which are disclosed in European Patent Application No. 0,040,696 of Baldwin, Bolhofer, Lumma, Amato, Karady & Weinstock, published Dec. 2, 1981. Preferred is L-643728, 4-amino-3-(2-(5-(dimethylaminomethyl)-2-furanymethylthio) ethylamino)-5-thoxycarbonylisothiazole- 1,1-dioxide.

Selective H-2 antagonists include the 2-substituted amino-4(1H)-pyrimidone derivatives meeting the above criteria which are disclosed in European Patent Application No. 0,186,275 of Yanagisawa, Ohta, Takagi & Takeuchi, published Jul. 2, 1986. Preferred is HB-408, 5-butyl-6-methyl-2-(3-(3-(piperidinomethyl) phenoxy)propylamino)pyrimidin-4(1H)-one.

II. Gastrin Receptor and Gastrin Receptor Antagonists

Cholecystokinin (CCK) is a gastrointestinal hormone which is produced by and released from duodenal and jejunal mucous membranes, and is known to have actions such as secretion of pancreatic juice, gallbladder constriction, and stimulation of insulin secretion. CCK is also known to be present in the cerebral cortex, hypothalamus, and hippocampus at a high concentration and exhibit actions such as inhibition of eating and hunger, augmentation of memory, and generation of anxiety. Gastrin is a gastrointestinal hormone which is produced by and released from G-cells distributed in the pylorus and is known to exhibit actions such as secretion of gastric acid and constriction of the pylorus and gallbladder. CCK and gastrin, having the same five amino acids in their C-terminals, express actions via receptors. CCK receptors are classified into CCK-A which are peripheral type receptors distributed in the pancreas, gallbladder, and intestines; and CCK-B which are central type receptors distributed in the brain. Since gastrin receptors and CCK-B receptors show similar properties in receptor-binding tests and have high homology, they are often called CCK-B/gastrin receptors.

Compounds having antagonism to these receptors, for example, gastrin or CCK-B receptor, are useful for prevention or treatment of gastric ulcer, duodenal ulcer, gastritis, reflux esophagitis, pancreatitis, Zollinger-Ellison syndrome, vacuolating G-cell hyperplasia, basal-mucous-membrane hyperplasia, cholecystitis, attack of biliary colic, dysmotilities of alimentary canal, irritable bowel syndrome, certain types of tumors, eating disorders, anxiety, panic disorder, depression, schizophrenia, Parkinson's disease, tardive dyskinesia, Gilles de la Tourette syndrome, drug dependence, and drug- withdrawal symptoms. Moreover, the compounds are expected to induce pain relief or to accelerate induction of pain relief by opioid medications (Folia Pharmacologica Japonica, Vol. 106, 171-180 (1995), Drugs of the Future, Vol. 18. 919-931 (1993), American Journal of Physiology, Vol. 269, G628-G646 (1995), American Journal of Physiology, Vol. 259, G184-G190 (1990), European Journal of Pharmacology, 261, 257-263 (1994), Trends in Pharmacological Science, Vol. 15, 65-66 (1994)).

As stated above, gastrin is known to be largely responsible for controlling histamine production, e.g., by the ECL cells in the stomach. At least certain aspects of the present invention are based on the belief that, by blocking the gastrin receptor, less histamine is produced and thus the effect should be similar to that of blocking the histamine-2 receptor directly.

The following teachings relate to classes of chemical antagonists, i.e., small molecule antagonists of the CCK/gastrin receptors. However, it will be appreciated by the skilled artisan that any antagonist of the CCK/gastrin receptors, including but not limited to, proteins, nucleic acids, carbohydrates, lipids or any other molecules that bind or interact with the CCK/gastrin receptors can be utilized in the combination therapies described herein. Moreover, indirect means of antagonizing CCK/gastrin receptors are known in the art and can be used in the combination therapies described herein.

Several classes of CCK receptor antagonists have been reported in the literature. One class comprises derivatives of cyclic nucleotides, for example, dibutyryl cyclic GMP. Another art recognized class of CCK antagonists comprise the C-terminal fragments and analogs of CCK. Another class of CCK receptor antagonists are amino acid derivatives including proglumide, a derivative of glutaramic acid, and the N-acyltryptophanes such as p-chlorobenzoyl-L-tryptophan. More recently certain substituted amino phenyl compounds were described as CCK antagonists in published European Patent Application 0166355. Because of the wide range of potential clinical applications of CCK binding compounds, intensive research efforts have been ongoing to define other compounds exhibiting CCK receptor binding properties.

Preferred CCK-B/gastrin receptor antagonists include, but are not limited to: L365,260; L740,093 (Merck); CI-988 (formerly PD-134,308; Parke-Davis); CAM-1028; CI-1015; PD135158; PD136450; PD140,376; GV150013X (Glaxo-Wellcome); LY288513 (Lilly); YM022 (Yamanouchi, Inc., Japan); YF476 (Ferring Research Institute/Yamanouchi); JB93182 (James Black Foundation); RP73 870; RPR- 101048; RB213; AG041R; DA-3934 (Daiichi Pharmaceutical); CR 2945 (see for example: Li Y, et al., American Journal of Physiology, 1999, 277(2 Pt 1):G469-77; Goddard A W, et al., Psychiatry Research, 1999, 85(3):225-40; Wiesenfeld-Hallin Z., et al., Behavioral & Brain Sciences, 1997, 20(3):420-5; discussion 435-513; Sandvik A K, and Dockray G J., European Journal of Pharmacology, 1999, 364(2-3):199-203; Kajbaf M, et al., Xenobiotica, 1998, 28(8):785-94; Luo B., et al., Brain Research, 1998, 796(1-2):27-37; Brenner L A, and Ritter R C., Physiology & Behavior 1998, 63(4):711-6; Trivedi B K., et al., Journal of Medicinal Chemistry, 1998, 41(1):38-45; Smadja C., et al., Psychopharmacology, 1997, 132(3):227-36; Rasmussen K., et al., Neuroscience Letters, 1997, 222(1):61-4; Semple G., et al., Journal of Medicinal Chemistry, 1997, 40(3):331 -41; Lena I., et al., Journal of Neurochemistry, 1997, 68(1):162-8; Patel S., et al., Regulatory Peptides, 1996, 65(1):29-35; Hirst G C., et al., Journal of Medicinal Chemistry, 1996, 39(26):5236-45; Horwell D., et al., Immunopharmacology, 1996, 33(1-3):68-72; Helton D R., et al., Pharmacology, Biochemistry & Behavior, 1996, 53(3):493-502; Rasmussen K., et al., Neuroreport, 1996, 7(5):1050-2; Araldi G., et al., Farmaco, 1996, 51(7):471-6; Weng J H., et al., Bioorganic & Medicinal Chemistry, 1996, 4(4):563-73; Goudreau N., et al., Archiv der Pharmazie, 1996, 329(4):197-204).

Preferred gastrin receptor antagonists are of the formula (I): 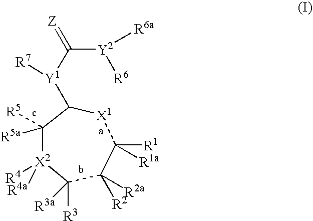
wherein

a, b, and c are each independently a single or double bond;

X¹, X², Y², and Y² are each independently carbon, nitrogen, oxygen or sulfur;

Z is oxygen, sulfur or nitrogen;

R¹ and R^{1a} are each independently hydrogen, alkyl, alkyloxy, alkylcarboxy, alkenyl, alkynyl, aryl, heteroaryl, amino, alkylamino, amido, alkylamido, nitro, cyano, hydroxy, halogen or R^{1a} is absent if a is a double bond;

R², R^{2a}, R³, and R^{3a} are each independently hydrogen, alkyl, alkyloxy, alkylcarboxy, alkenyl, alkynyl, aryl, heteroaryl, or R^{2a} and R^{3a} are absent if b is a double bond, or R² and R³ may be linked to form a ring;

R⁴ and R^{4a} are each independently hydrogen, alkyl, alkyloxy, alkylcarboxy, alkenyl, alkynyl, alkylaryl, amino, alkylamino, alkylamido, R^{4a} and R⁴ are absent if X² is oxygen or sulfur, or R^{4a} is absent if X² is nitrogen;

R⁵ and R^{5a} are each independently hydrogen, alkyl, alkyloxy, alkylcarboxy, alkenyl, alkynyl, aryl, heteroaryl, amino, alkylamino, amido, alkylamido, mercapto, alkyl mercapto, nitro, cyano, hydroxy, halogen or R^{5a} is absent if c is a double bond;

R⁶ and R^{6a} are each independently hydrogen, alkyl, alkenyl, alkynyl, aryl, acyl heteroaryl, or R^{6a} is absent if Y² is oxgen;

R⁷ is hydrogen, alkyl, alkenyl, alkynyl, aryl, heteroaryl, acyl, or absent if Y¹ is oxygen, or pharmaceutically acceptable salts, esters or prodrugs thereof.

In an exemplary embodiment, Y¹ and Y² are nitrogen. In another exemplary embodiment, R^{6a} and R⁷ are hydrogen. In yet another exemplary embodiment, Z is oxygen.

Additional preferred gastrin receptor antagonists are of the formula (II): 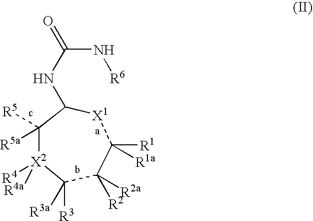
wherein

a, b, and c are each independently a single or double bond;

X¹ and X² are each independently carbon, nitrogen, oxygen or sulfur;

Z is oxygen, sulfur or nitrogen;

R¹ and R^{1a} are each independently hydrogen, alkyl, alkyloxy, alkylcarboxy, alkenyl, alkynyl, aryl, heteroaryl, amino, alkylamino, amido, alkylamido, nitro, cyano, hydroxy, halogen or R^{1a} is absent if a is a double bond;

R², R^{2a} , R³, and R^{3a} are each independently hydrogen, alkyl, alkyloxy, alkylcarboxy, alkenyl, alkynyl, aryl, heteroaryl, R^{2a} and R^{3a} are absent if b is a double bond, or R² and R³ may be linked to form a ring;

R⁴ and R^{4a} are each independently hydrogen, alkyl, alkyloxy, alkylcarboxy, alkenyl, alkynyl, alkylaryl, amino, alkylamino, alkylamido, R^{4a} and R⁴ are absent if X² is oxygen or sulfur, or R^{4a} is absent if X² is nitrogen;

R⁵ and R^{5a} are each independently hydrogen, alkyl, alkyloxy, alkylcarboxy, alkenyl, alkynyl, aryl, heteroaryl, amino, alkylamino, amido, alkylamido, mercapto, alkyl mercapto, nitro, cyano, hydroxy, halogen or R^{5a} is absent if c is a double bond; and

R⁶ is hydrogen, alkyl, alkenyl, alkynyl, aryl, or heteroaryl.

In one exemplary embodiment, X¹ and X² are nitrogen and R^{4a} is absent. In another exemplary embodiment, a is a double bond and R^{1a} is absent. In yet another exemplary embodiment, c is a double bond and R^{5a} is absent.

Additional preferred gastrin receptor antagonists are of the formula (III): 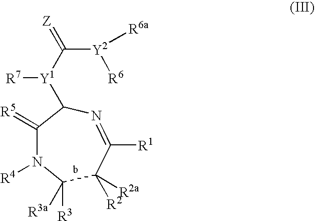
wherein:

b is a single or double bond;

Y¹ and Y² are each independently carbon, nitrogen, oxygen or sulfur;

Z is oxygen, sulfur or nitrogen;

R¹ is hydrogen, alkyl, alkyloxy, alkylcarboxy, alkenyl, alkynyl, aryl, heteroaryl, amino, alkylamino, amido, alkylamido, nitro, cyano, hydroxy, halogen or R^{1a} is absent if a is a double bond;

R², R^{2a}, R³, and R^{3a} are each independently hydrogen, alkyl, alkyloxy, alkylcarboxy, alkenyl, alkynyl, aryl, heteroaryl, R^{2a} and R^{3a} are absent if b is a double bond, or R2 and R³ may be linked to form a ring;

R⁴ is hydrogen, alkyl, alkyloxy, alkylcarboxy, alkenyl, alkynyl, alkylaryl, amino, alkylamino, alkylamido;

R⁵ is oxygen, sulfur, —CR^{8a}R^{8b}, or —NR^{8a};

R⁶ and R^{6a} are each independently hydrogen, alkyl, alkenyl, alkynyl, aryl, acyl heteroaryl, or R^{6a} is absent if Y² is oxgen;

R⁷ is hydrogen, alkyl, alkenyl, alkynyl, aryl, heteroaryl, acyl, or absent if Y¹ is oxygen; and

R⁸ is hydrogen, alkyl, alkenyl, alkynyl, cycloalkyl, aryl, or heteroaryl.

In one exemplary embodiment, b is a double bond and R^{2a} and R^{3a} are absent. In another exemplary embodiment, R² and R³ are linked to form an alkyl or aryl ring.

Additional preferred gastrin receptor antagonists are of the formula (IV): 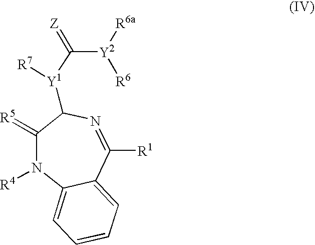
wherein:

Y¹ and Y² are each independently carbon, nitrogen, oxygen or sulfur;

Z is oxygen, sulfur or nitrogen;

R¹ is hydrogen, alkyl, alkyloxy, alkylcarboxy, alkenyl, alkynyl, aryl, heteroaryl, amino, alkylamino, amido, alkylamido, nitro, cyano, hydroxy, or halogen;

R⁴ is hydrogen, alkyl, alkyloxy, alkylcarboxy, alkenyl, alkynyl, alkylaryl, amino, alkylamino, alkylamido;

R⁵ is oxygen, sulfur, —CR^{8a}R^{8b}, or —NR^{8a};

R⁶ and R^{6a} are each independently hydrogen, alkyl, alkenyl, alkynyl, aryl, acyl heteroaryl, or R^{6a} is absent if Y² is oxgen;

R⁷ is hydrogen, alkyl, alkenyl, alkynyl, aryl, heteroaryl, acyl, or absent if Y¹ is oxygen; and

R⁸ is hydrogen, alkyl, alkenyl, alkynyl, cycloalkyl, aryl, or heteroaryl.

In one exemplary embodiment, The method Y¹ and Y² are nitrogen. In another exemplary embodiment, Z is oxygen. In another exemplary embodiment, R^{6a} is hydrogen. In another exemplary embodiment, R⁷ is hydrogen. In yet another exemplary embodiment, R⁵ is oxygen.

Yet other preferred gastrin receptor antagonists are of the formula (V): 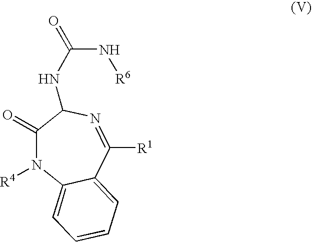
wherein

R¹ is hydrogen, alkyl, alkyloxy, alkylcarboxy, alkenyl, alkynyl, aryl, heteroaryl, amino, alkylamino, amido, alkylamido, nitro, cyano, hydroxy, or halogen;

R⁴ is hydrogen, alkyl, alkyloxy, alkylcarboxy, alkenyl, alkynyl, alkylaryl, amino, alkylamino or alkylamido; and

R⁶ is hydrogen, alkyl, alkenyl, alkynyl, cycloalkyl, aryl, or heteroaryl.

In one exemplary embodiment, R¹ is a 2,3, or 4-pyridyl. In another exemplary embodiment, R⁴ is acetonyl. In yet another exemplary embodiment, R⁶ is ortho-, meta-, orpara-N-methylaniline.

Yet another preferred gastrin receptor antagonist is:

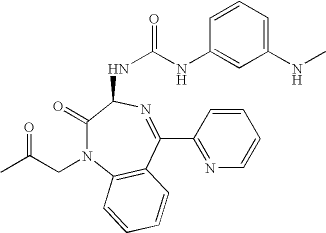

While the exemplification set forth herein features the use of antagonists specific for the CCK-B receptor, the skilled artisan will appreciate that antagonizing other classes of CKK receptors, in combination with histamine 2-receptor antagonism, is also within the scope of the invention. The skilled artisan will also appreciate that the instant combination therapy methods would be applicable in the case of heretofore unidentified CCK and/or gastrin receptors, in particular with regards to antagonizing such receptors in combination with histamine antagonism.

III. Methods of Treatment

The invention further relates to a method for treating or preventing a gastrointestinal disorder, e.g., an acid peptic disorder, by administering at least two agents, each of which is a compound that contributes to the therapeutic effect when co-administered and is useful in treating or preventing the disorder. The first compound of the invention is a Histamine 2- receptor antagonist that are useful for treating or preventing an acid peptic disorders. The second compound is a Gastrin receptor antagonist (CCK, CCK-B, CCK2RA) that are useful for treating or preventing an acid peptic disorders.

The effectiveness of this combination therapy has been demonstrated in an animal model of gastic cancer. In particular, studies were designed which involved testing the effects of loxtidine and CCK2RAs (YF476) on a mouse model of gastric cancer. This mouse model involved infecting insulin-gastrin (INS-GAS) transgenic mice with the organism Helicobacter felis, and the mice develop an accelerated cancer over a period of 6-7 months post infection. This mouse model demonstrates a clear role for gastrin in the induction of gastric cancer. Given this model and human data suggesting that PPIs can accelerate atrophy, it was expected that loxtidine, by inhibiting acid and inducing hypergastrinemia, would accelerate further cancer in the model. Surprisingly, however, it was found that both loxtidine and the CCK2R antagonist (YF476) partially blocked the development of gastric hyperplasia/preneoplasia, and that the combination completely inhibited the development of epithelial changes in the INS-GAS/H. felis mouse model.

Both loxtidine and YF476 individually resulted in an increase in body weight (from 28 to 33 gm) and a reduction in stomach weight (from 1831 to ˜400 mg). However, the combination of loxtidine and YF476 resulted in a decrease in stomach weight to essentially normal (320 mg). In addition, the two agents showed strong synergy in inhibiting acid secretion in the mouse model. While loxtidine reduced gastric acid output by 83% and YF476 reduced gastric acid output by 91%, the combination reduced gastric acid secretion by 100%. This is the most effective therapy reported to date in an animal model for blocking acid secretion, and it was accomplished without any of the adverse growth effects reported for most other compounds.

Based on these findings, it is proposed that the combination of an H2RA/CCK2RA will make the ideal agent for treatment of acid peptic disorders and prove superior to any other agent or combination. This is particularly the case if loxtidine is utilized as the H2RA because it is long lasting and an irreversible H2RA, while others (cimetidine, ranitidine) are reversible inhibitors with shorter duration of action. Blocking histamine signaling is beneficial as histamine is a downstream “growth factor” that mediates much of gastrin's undesirable effects on the mucosa.

The combination therapy preferably has the effect of diminishing specific symptoms which are characteristic of acid peptic disorders (e.g. heartburn, dyspepsia etc.). The first and second compounds may exert their biological effects by similar or unrelated mechanisms of action; or either one or both of the first and second compounds may exert their biological effects by a multiplicity of mechanisms of action. A third compound, or even more yet, may likewise be used in a method of the invention, wherein the third (and fourth, etc.) compound has the same characteristics of a second compound.

The combination therapies of the invention feature administration of a therapeutically effective amount of a histamine 2-receptor antagonist and a gastrin receptor antagonist. An effective amount is the dosage of each agent sufficient to provide the medically desirable result, when administered in combination. The effective amount of the agents will vary with the particular condition being treated, the age and physical condition of the subject being treated, the severity of the condition, the duration of the treatment, the nature of the concurrent therapy (if any), the specific route of administration and like factors within the knowledge and expertise of the health practitioner.

It should be understood that the agents of the invention are used to reduce the risk of developing, or to treat gastrointestinal disorders (e.g., peptic acid disorders), that is, they are used prophylactically in subjects at risk of developing a gastrointestinal disorder (asymptomatic), and acutely in subjects already symptomatic for the disorder. Thus, the effective amount is that amount which can lower the risk of, slow, reverse, or perhaps prevent altogether the development of a gastrointestinal disorder (e.g., peptic acid disorders). It will be recognized that when the agent is used in acute circumstances, it is usually used to prevent one or more medically undesirable results. In the case of gastric atrophy, the agents can be used to limit parietal cell loss and/or prevent the atrophy from progressing to gastric cancer.

Generally, doses of active compounds are from about 0.001 mg/kg per day to 1000 mg/kg per day of each agent. Doses ranging from 50-500 mg/kg would also be suitable, preferably orally and in one or several administrations per day. Lower doses will result from other forms of administration, such as intravenous administration. Ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight, are exemplary. In the event that a response in a subject is insufficient at the initial doses applied, higher doses (or effectively higher doses by a different, more localized delivery route) may be employed to the extent that patient tolerance permits. Agents can be administered daily, every two, three, four, five or six days, weekly, etc. Multiple doses per day are also contemplated to achieve appropriate systemic levels of compounds.

Exemplary doses of H2RA (e.g., loxtidine) are within the range of 0.01 to 1.0 mg/kg/day, preferably within the range of about 0.02 to 0.5 mg For example, in the treatment of gastric cancer, the dose given for the H2RA (e.g., loxtidine) can be about 0.1 mg/kg/day, or about 7 mg per day administered to a 70 kg patient. The CCK-B antagonists, (e.g., YF476 and YM022) can be administered at doses ranging from 50 nmol/kg/day to 25 micromol/kg/day (Ding et al, Pharmacology & Toxicology 1997;81:232-237). An exemplary dose for YF476 is within the range of about 0.5 to 50 mg/kg/day, preferably within the range of about 1 to 25 mg/kg/day. A preferred dose for YF476 is about 80 micromole/kg/week or 5.7 mg/kg/day (i.e., about 400 mg/day for a 70 kg patient). The ID₅₀ for YF476 is about 50 nmol/kg/day which results in 50% inhibition of the CCK-B receptor effect. YM022 is reportedly about 5 times more potent than YF476 (Lindstrom et al, British Journal of Pharmcology, 1999, 127:530-536).

IV. Pharmaceutical Compositions and/or Formulations

Advantageous pharmaceutical compositions of the invention are formulated to be orally administered to a subject. The first agent and said second agent may be simultaneously administered. The first agent and the second agent may modulate different biological processes of acid peptic disorders. The first agent and the second agent can act on different targets. An additional agent may therapeutically useful in reducing or inhibiting cellular toxicity. The first agent and the second agent may have different binding affinities or specificities for peptides, proteins, or enzymes involved in the pathogenesis of acid peptic disorders. The first agent and the second agent, when simultaneously present in a subject, act synergistically to reduce, inhibit, or ameliorate the symptoms of acid peptic disorders. The invention also relates to the use of a first agent and a second agent in the preparation of a pharmaceutical composition for the treatment or prevention of an acid peptic disease comprising a first agent and a second agent in a pharmaceutically acceptable carrier, wherein the first agent prevents or inhibits or cellular toxicity; and the second agent is a therapeutic agent which inhibits gastric acid secretion.

A variety of pharmaceutically-acceptable carriers may be included, depending on the particular dosage form to be used. Various oral dosage forms can be used, including such solid forms as tablets, capsules, granules and bulk powders. Tablets can be compressed, tablet triturates, enteric-coated, sugar-coated, film-coated or multiple compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules and effervescent preparations reconstituted from effervescent granules, containing suitable solvents, preservatives, emulsifying agents, suspending agents, diluents, sweeteners, melting agents, coloring, and flavoring agents.

Some examples of substances which can serve as pharmaceutically-acceptably carriers are sugars such as lactose, glucose and sucrose; starches such as corn starch and potato starch; cellulose and its derivatives such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobroma; polios such as propylene glycol, glycerine, sorbitol, mannitol, and polyethylene glycol; agar; agonic acid; progeny-free water; isotonic saline; and phosphate buffer solutions, as well as other non-toxic compatible substances used in pharmaceutical formulations. Wetting agents and lubricants such as sodium laurel sulfate, as well as coloring agents, flavoring agents, recipients, tabulating agents, stabilizers, anti-oxidants, and preservatives can also be present. Other compatible pharmaceutical additives and actives (e.g., NSAI drugs; pain killers; muscle relaxants) may be included in the pharmaceutically-acceptable carrier for use in the compositions of the present invention.

The choice of a pharmaceutically-acceptable carrier to be used in conjunction with the CCK2R and H2RA combination of the present invention is basically determined by the way the composition is to be administered. The preferred mode of administering the compositions of the present invention is orally. The preferred unit dosage form is therefore tablets, capsules, and the like, comprising a safe and effective amount of the CCK2R and H2RA combination of the present invention. Pharmaceutically-acceptable carriers suitable for the preparation of unit dosage forms for oral administration are well known in the art. Their selection will depend on secondary considerations like taste, cost, shelf stability, which are not critical for the purposes of the present invention, and can be made without difficulty by a person skilled in the art.

Additional routes of administration are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral (described in detail above), rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency or acute situations. Oral administration will be preferred for prophylactic treatment because of the convenience to the patient as well as the dosing schedule.

Compositions suitable for parenteral administration conveniently comprise a sterile aqueous preparation of the anti-inflammatory agent, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can further be found in Remington's Pharmaceutical Sciences, Mack Publishing Co., Easton, Pa.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189 and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention also relates to packaged pharmaceutical products containing two agents, each of which exerts a therapeutic effect when administered to a subject in need thereof, and is useful in treating or preventing an acid peptic disorder. The first agent of a pharmaceutical composition of the invention is selected from an H2RA that are useful for treating or preventing an acid peptic disease. The second agent is a CCK2RA may be useful in treating or preventing an acid peptic disorder. Either one or both agents (or optional additional agents) may further be useful in inhibiting or reducing cellular toxicity. The agents may exert their biological effects by similar or unrelated mechanisms of action; or either one or more than one of the agents may exert their biological effects by a multiplicity of mechanisms. A pharmaceutical composition may also comprise a third agent, or even more agents yet, wherein the third (and fourth, etc.) agent has the same characteristics of a second agent. In some cases, the individual agents may be packaged in separate containers for sale or delivery to the consumer. The agents of the invention may be supplied in a solution with an appropriate solvent or in a solvent-free form (e.g., lyophilized). Additional components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The present invention also includes packaged pharmaceutical products containing a first agent in combination with (e.g., intermixed with) a second agent. The invention also includes a pharmaceutical product comprising a first agent packaged with instructions for using the first agent in the presence of a second agent or instructions for use of the first agent in a method of the invention. The invention also includes a pharmaceutical product comprising a second or additional agents packaged with instructions for using the second or additional agents in the presence of a first agent or instructions for use of the second or additional agents in a method of the invention. Alternatively, the packaged pharmaceutical product may contain at least one of the agents and the product may be promoted for use with a second agent. It is also within the scope of the invention to administer, or promote (for administration) a first compound to a subject having the second agent already in his or her system, for example, as a result of a previous or concomitant therapy. Alternatively, the invention encompasses administration (or promotion for administration) of a second compound to a subject already having in his or her system the first agent, for example as a result of a previous or concomitant therapy.

### EXAMPLES

The invention is further illustrated by the following examples which should not be construed as limiting.

Materials and Methods

Animals

The insulin-gastrin (INS-GAS) transgenic mice (FVB/N background) have been described previously (Wang, T. C. et al., 2000; Wang T. C. et al., 1993) and were free of specific pathogens. Animals were housed in microisolator, solid-bottomed polycarbonate cages, fed a commercially prepared pelleted diet, and given water ad libitum. One hundred twenty four (124) male INS-GAS mice at 2 or 3 months of age were inoculated with Helicobacter felis (ATCC 49179) as previously described (Wang, T. C. et al., 2000). Infection status was confirmed at 14 weeks and 26 weeks post inoculation by enzyme-linked immunosorbent assay (ELISA) to measure immunoglobulin IgG antibody to H. felis, and by quantitative real-time PCR assays of gastric corpus tissue at necropsy as described below. All experiments were approved by the Institutional Animal Care and Use Committee of University of Massachusetts Medical School.

Drugs and experimental design

The CCK-B/gastrin receptor antagonist YF476 was a kind gift of Dr. Keiji Miyata and Dr. Hidenobu Yuki (Yamanouchi Pharmaceutical Co. Ltd., Tsukuba, Japan) (Takinami, Y. et al., 1997). The drug was dissolved in PEG300 at the concentration of 12 mg/ml and subcutaneously injected every week at the dose of 40 mg/kg(=80 micromole/kg). The irreversible histamine H2 receptor antagonist loxtidine was manufactured by GlaxoSmithKlein (Research Triangle Park, N.C.) and was a kind gift of Prof. Duan Chen and Prof. Rolf Hakanson. The drug was dissolved in sterilized drinking water at the concentration of 0.5 gram/liter and given to the mice ad libitum as previously described (Kidd, M. et al., 2000). Loxtidine-containing water bottles were changed weekly, and consumed water volume was measured for each bottle.

One hundred (100) male INS-GAS mice with H. felis infection were divided into four groups, and treated with YF476 and loxtidine for 3 or 6 months. Mice in group I were controls treated with vehicle only, whereas group II mice were subcutaneously injected with the gastrin/CCK-B receptor antagonist YF476 at a dose of 80 micromole/kg once per week. Mice in group III were treated orally with the histamine H2 receptor antagonist loxtidine (in drinking water) at a dose of 0.5 g/L, while group IV mice received both drugs. For the 3 months drug study of inhibitory effects on maximal acid secretion, gastric atrophy, hyperplasia and dysplasia, 8 mice were prepared for each group. For the 6 months drug study of inhibitory effects on gastric carcinogenesis, 17 mice were prepared for each group.

The proton pump inhibitor omeprazole was purchased from Sigma (St. Louis Mo.) and dissolved in dimethylsulfoxide (DMSO)/PEG300, a 1:1 mixture at the concentration of 40 mg/ml, and injected intraperitoneally daily at the dose of 4 mg/mouse (=350 micromole/kg) for 3 months. Twenty four (24) mice were divided into four groups. Mice in group A were controls treated with vehicle only, whereas group B mice were treated with omeprazole alone. Group C and D mice were treated with combination of omeprazole and YF476 or loxtidine, respectively.

Histological evaluation

At necropsy, linear strips extending from the squamocolumnar junction through the proximal duodenum were fixed in 10% neutral-buffered formalin, paraffin-embedded, cut at 5 microns, and stained with hematoxylin and eosin. Indices of injury in the gastric cardia/corpus and antrum were scored on an ordinal scale from 0 to 4 in increments of 0.5 by a single veterinary pathologist (A.B.R.) blinded to treatment groups. Paraffin-embedded sections were also stained with the Warthin-Starry silver staining method for detection of H.felis as previously described (Fox, J. G. et al., 2003).

Measurement of maximal gastric acid output

The pyloric ligation method was used to measure maximal acid secretion as previously described (Chen, D. et al., 2004). Briefly, mice were fasted overnight and anesthetized by isoflurane inhalation. The abdomen was incised by midline celiotomy, the pylorus ligated firmly, and the abdomen closed with surgical sutures. After 4 hours, the mice were euthanized and gastric juice collected. The acidity of gastric juice was measured with a pH meter (AR 25; Fisher Scientific, Houston, Tex.) by 0.01 N NaOH titration and results expressed as μEq.

Evaluation of serum antibody responses to H. felis

Serum was collected at 3 and 6 months post infection and evaluated by ELISA for serum IgG2a and IgG1 using an outer membrane antigen preparation of H. felis as previously described (Fox, J. G., et al., 2000; Fox, J. G. et al., 2003). Antigen was coated overnight at 4° C. on Immulon II plates (Thermo Labsystems, Franklin, Mass.) plates at a concentration of 10 μg/ml and sera were diluted 1:400. Biotinylated secondary antibodies included monoclonal anti-mouse antibodies produced by clones G1-6.5 and R19-157 (Pharmingen, San Diego, Calif.) for detecting IgG1 and IgG2a, respectively. Incubation with extravidin peroxidase (Sigma) was followed by ABTS® substrate (Kirkegaard and Perry Laboratories, Gaithersburg, Md.) for color development. Optical density (OD) development at 405λ was recorded by an ELISA plate reader (Dynatech MR7000, Dynatech Laboratories, Inc., Chantilly, Va.).

Gastrin Radioimmunoassay

Plasma gastrin levels (COOH-terminally amidated gastrin) were determined by radioimmunoassay using rabbit antiserum L2 that reacts similarly with G17 and G347. Real-time PCR Assay of Helicobacter felis infection in mouse stomachs and RT-PCR assay of growth factors and cytokine expression profiles H.felis DNA present in infected mouse stomachs was quantified using a modification of a previously described realtime PCR assay that accurately quantified H. pylori (Ge, Z. et al., 2001). Two primers (forward: 5′-TTCGATTGGTCCTACAGGCTCAGA -3′ (SEQ ID NO:1); reverse: 5′-TTCTTGRRGFATGACATTGACCAACGCA-3′(SEQ ID NO:2) were designed to hybridize within the conserved region of the single copy H. felis flaB gene locus. No products were amplified from DNA isolated from H. pylori, H. mustelae, or H. bizzozeroni, confirming the specificity of these oligonucleotides for H. felis. DNA from plate-grown H. felis and from mouse gastric corpus was prepared using a high pure PCR kit (Roche Molecular Biochemicals, Indianapolis, IN). Real-time PCR was performed using SmartCycler (Cepheid, Calif.) and Quantitect SYBR Green PCR kit (QIAGEN Inc., Valencia, Calif.) following the manufacturer's instructions. Briefly, the PCR assay was performed under the following conditions: 95° C. for 15 minutes followed by 45 cycles of 95° C. for 15 seconds, 55° C. for 30 seconds and 72° C. for 30 seconds. Ten-fold dilutions (5×106 to 5×103 copies) of DNA from H. felis were used to generate a standard curve, and serially diluted standards were simultaneously amplified with in vivo samples of mice gastric corpus DNA (100 ng per sample), and H. felis mucosal DNA copy numbers were then normalized per stomach DNA (copies/μg) (Fox, J. G. et al., 2003; Ge, Z. et al., 2001).

Real-time RT-PCR assay of growth factors and cytokine expression profiles Total RNAs were extracted from a sample of whole stomach from each animal with Trizol (Invitrogen, Calif.) and five micrograms of total RNA were used for first strand cDNA synthesis using Superscript II cDNA amplification System (Invitrogen, Calif.) following manufacturer's instructions. Real-time PCR was performed as above using RT-PCR primers for each gene as listed below. All primers were designed using Lasergene ver5.0 software (DNASTAR inc., Madison, Wis.). Results were calculated by minus delta delta threshold cycle (-ΔΔCt) method (Livak, K. J. et al., 2001). Briefly, the threshold cycle Ct1 of each sample reaction was deducted from the threshold cycle Ct2 of GAPDH reaction for normalization, and then deducted from the threshold cycle Ct3 of calibration control (45 cycles in this experiment), i.e., the final result was represented by the formula; Ct3-(Ct1-Ct2). Reg I: forward 5′-aaggagagtggcactacagacg-3′(SEQ ID NO: 3), reverse 5′-gtattgggcatcacagttgtca-3′ (SEQ ID NO:4); HB-EGF: forward 5′-gacccatgcctcaggaaataca-3′(SEQ ID NO:5), reverse 5′-tacagccaccacagccaagact-3′(SEQ ID NO:6); Amphiregulin: forward 5′-ggcaaaaatggaaaaggcagaa-3′(SEQ ID NO:7), reverse 5′cgaggatgatggcagagacaaa-3′(SEQ ID NO:8); TGF-alpha: forward 5′gccggtttttggtgcaggaaga-3′ (SEQ ID NO:9), reverse 5′-ttgcggagctgacagcagtgga-3′(SEQ ID NO:10); IFNgamma: forward 5′-catggctgtttctggctgttactg-3′(SEQ ID NO:11), reverse 5′-gttgctgatggcctgattgtcttt-3′(SEQ ID NO: 12); TNF-alpha: forward 5′tggcccagaccctcacactcag-3′(SEQ ID NO:13), reverse 5′-acccatcggctggcaccact-3′(SEQ ID NO:14); IL-4: forward 5′-atcggcattttgaacgaggtca-3′ (SEQ ID NO:15), reverse 5′-catcgaaaagcccgaaag-3′(SEQ ID NO: 16); Somatostatin: forward 5′-gtcctggctttgggcggtgtca-3′(SEQ ID NO: 17), reverse 5′-tgcagctccagcctcatctcgt-3 (SEQ ID NO:18).

Statistical Analysis

The results are expressed as mean +SD unless otherwise stated. The Student t-test or Mann-Whitney test were used to evaluate statistical significance. Values of p<0.01 or p<0.05 were considered statistically significant.

### Example 1

### Testing the Effects of Loxtidine and YF476 on a Mouse Model of Gastric Cancer (INS-GAS/H. felis)

This Example describes the results of a study using a combination therapy of an H2RA and a CCK2RA for the treatment of acid peptic disorders in an animal model.

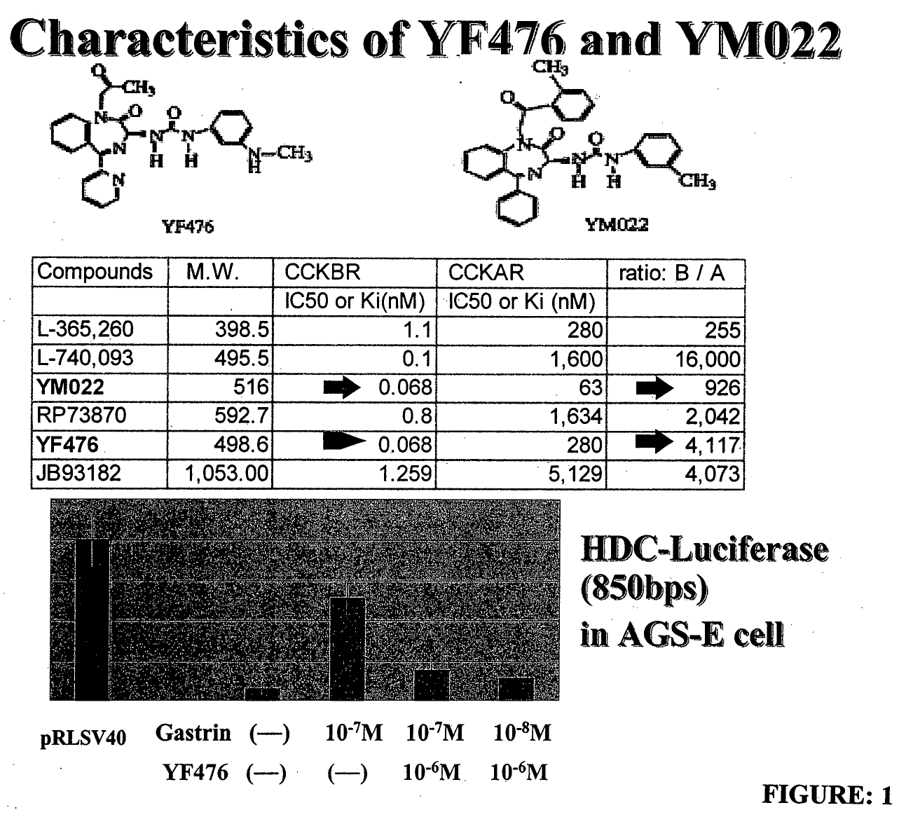

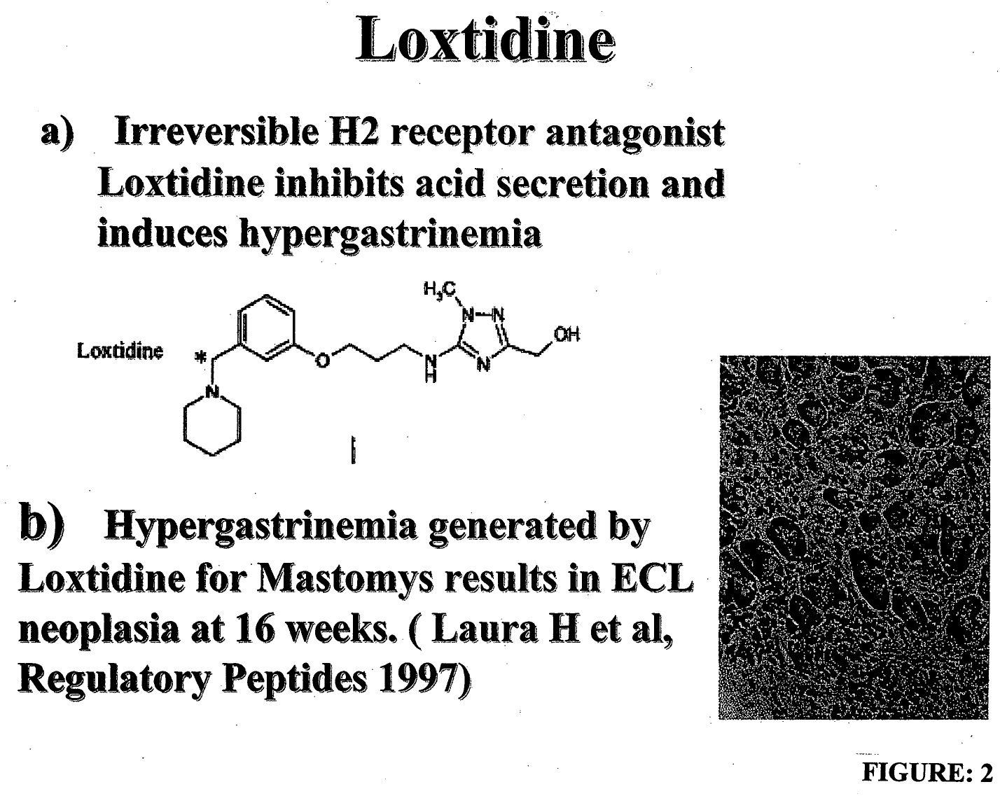

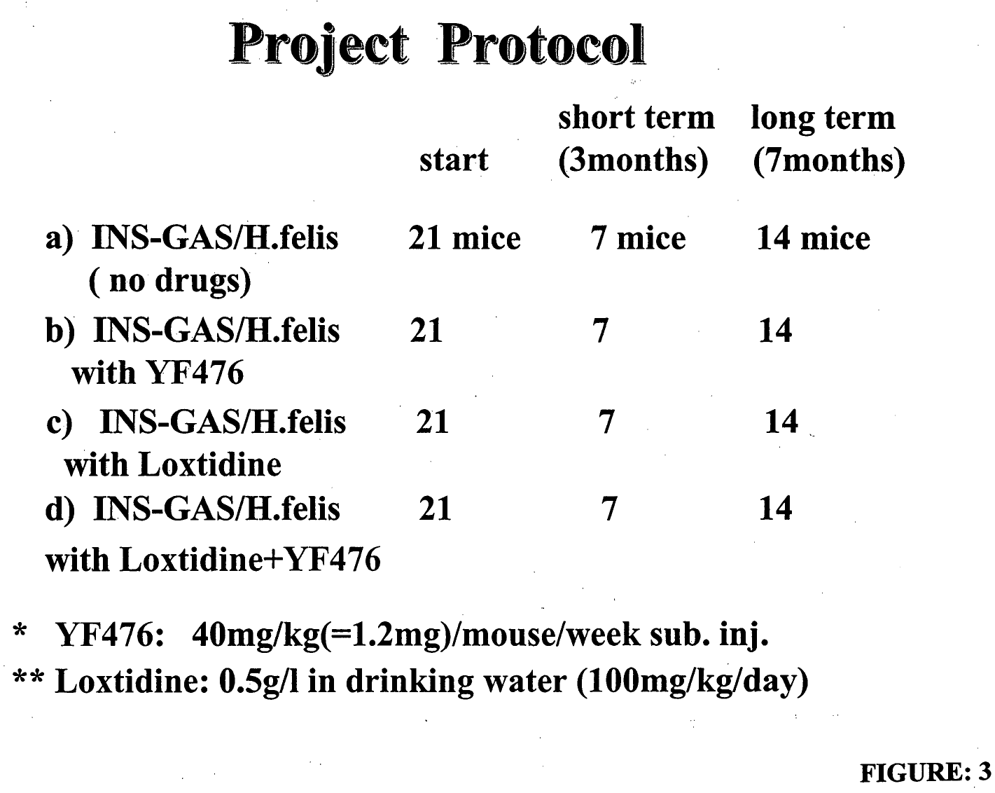

The H2RA, loxidine, and CCK2RA, YF476, were chosen for use in this study. The structural and functional characteristics of these compounds are set forth in FIGS. 1 and 2, respectively. The experimental protocol for this study is set forth in FIG. 3.

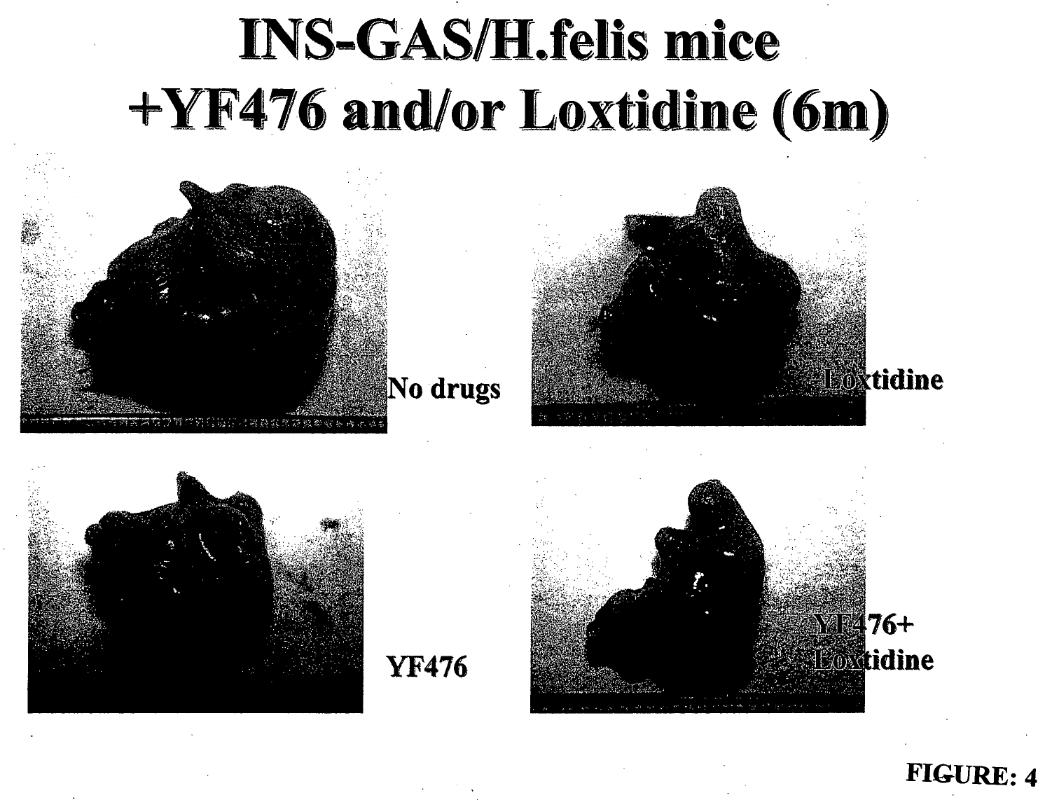

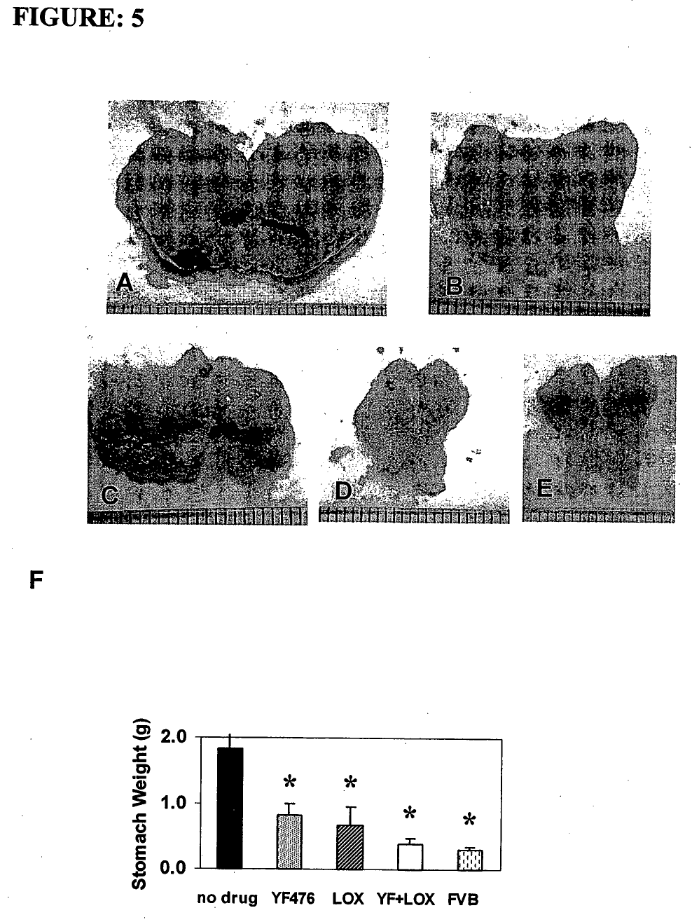

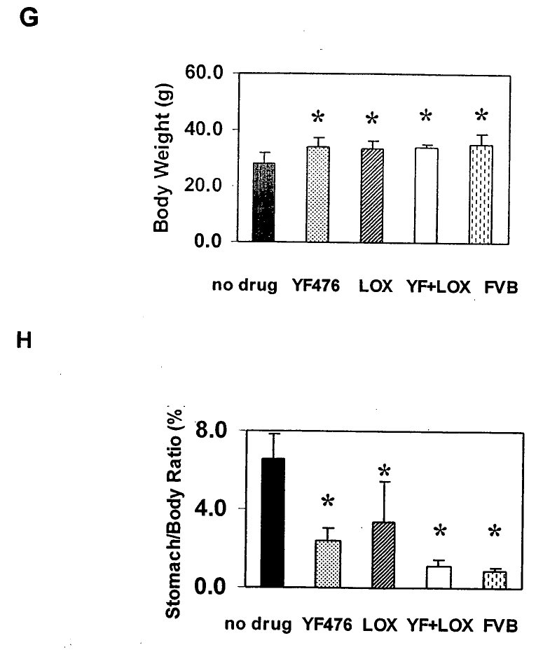

Both loxtidine and YF476 individually resulted in an increase in body weight (from 28 to 33 grams) and a reduction of stomach weight (from ˜1830 to ˜800mg or ˜475 mg, respectively). In particular, treatment with YF476 or loxtidine or both resulted in marked reduction in gross stomach size (FIG. 4) and also resulted in reduction in apparent size of gastric folds (FIG. 5B,C,D). The combination of loxtidine and YF476 resulted in a decrease in stomach weight to essentially normal (380 mg) (FIG. 5F).

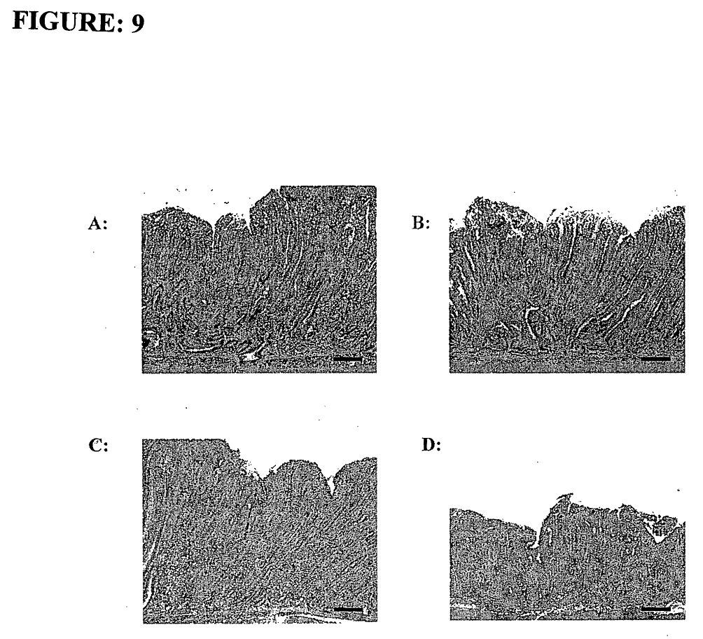

Loxtidine and the CCK2R antagonist (YF476) partially blocked the development of gastric hyperplasia/ preneoplasia and the combination completely inhibited the development of epithelial changes in the INS-GAS/H. felis mouse model. As shown in FIG. 9, the INS-GAS/H. felis mice at 3 months showed severe atrophy, metaplasia and foveolar hyperplasia. Treatment with YF476 resulted in a significant reduction in these changes, as did treatment with loxtidine (FIG. 9C,D). The combination of YF476 and loxtidine resulted in even greater inhibition of changes induced by Helicobadter felis in the INS-GAS mice, and resulted in a histopathologic appearance that was identical to the normal control (data not shown).

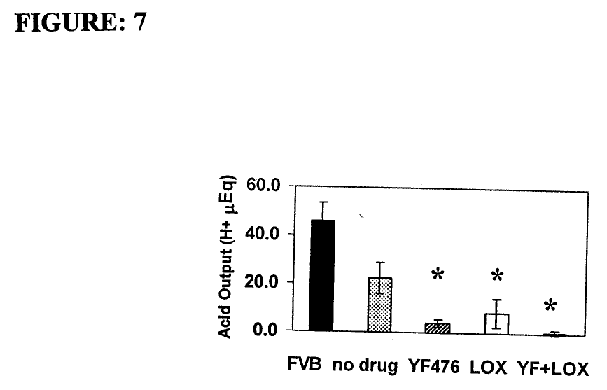

In addition, the two agents showed a strong synergy in inhibiting acid secretion in the mouse model. While loxtidine reduced gastric acid output by 83% and YF476 reduced gastric acid output by 91%, the combination reduced gastric acid secretion by 100% (FIG. 7).

In conclusion, this combination of an H2RA/CCK2RA makes an ideal agent for the treatment of acid peptic disorders and prove superior to other agent and/or combinations previously used to treat such disorders.

Introduction to Examples II-VII

Recent studies have confirmed that Helicobacter pylori infection represents the primary environmental risk factor for noncardia gastric cancer (Helicobacter and Cancer Collaborative Group (2001); Uemura, N. et al., 2001). Based on accumulated epidemiologic evidence, a Working Group of the International Agency for Research on Cancer (IARC), a branch of the World Health Organization, in 1994 classified H. pylori as a group I carcinogen (International Agency for Research on Cancer (1994)). Nevertheless, questions remain regarding the mechanisms by which Helicobacter pylori is able to promote neoplasia of the stomach. Data derived from both animal models and human studies have pointed to a host immune responses, particularly strong Th1 immune responses, as critical to the development of gastric atrophy and intestinal metaplasia, preneoplastic conditions strongly associated with progression to cancer (Houghton, J. et al., 2002; Fox J. G. et al., 2000). However, more recent studies in mice have suggested a role for hypergastrinemia in the pathogenesis of gastric cancer. Experimental studies on Hypergastrinemic mice (INS-GAS mice) show spontaneous development of gastric atrophy, metaplasia and adenocarcinoma which could be markedly accelerated by concurrent Helicobacter infection (Fox, J. G. et al., 2003).

These studies indicate that elevations in circulating gastrin-17 might directly promote gastric atrophy and preneoplasia, as well as, neoplasia of the stomach in a susceptible host. Nevertheless, the precise mechanism of action of gastrin and the critical downstream cellular targets of gastrin in this model have not been clearly defined. The decrease in parietal cells observed over time in hypergastrinemic mice may be a result of either decreased production or increased turnover of parietal cells. In addition, the effects of gastrin on the gastric mucosa may be either a direct effect (acting on cells expressing receptors) or an indirect effect (mediated by secondary growth factors). The effects of amidated gastrin (G-17) are clearly transduced by the CCK-B/gastrin receptor, a member of the larger G-protein coupled receptor (GPCR) family, and expression of the CCK-B receptor on both parietal and enterochromaffin-like cells of the stomach has clearly been demonstrated (Dockray, G. J. et al., 2001; Asahara, M. et al., 1994). Thus, gastrin exerts its proliferative effects at least partly indirectly through upregulation of paracrine growth factors such as HB-EGF (expressed in parietal cells) and Reg I (expressed in ECL cells). The role of gastrin directly exerting its effects on a progenitor cell population in the oxyntic glands has also been suggested (Kazumori, H. et al., 2001). However, one of the main targets of gastrin in the gastric mucosa is histamine production, given that histamine is the final common mediator of acid secretion. Hypergastrinemia acting on CCK-B receptors on ECL cells leads to both increased histamine release and increased histamine production (through upregulation of HDC) which stimulate acid secretion through H2 receptors on parietal cells. Nevertheless, the relative importance of histamine in the long-term mucosal response to hypergastrinemia in the INS-GAS mouse has not been directly addressed.

The role of hypergastrinemia in the development of gastric atrophy, particularly in human patients with or without H. pylori infection, has been controversial. It is generally accepted that infection with H. pylori results in a 1.5-2.0-fold elevation in serum gastrin-17 levels that occurs early in the course of infection, which precedes the development of atrophic gastritis, and typically resolves after eradication of infection. In addition, the majority of clinical studies have accepted that proton pump inhibitors (PPIs), which typically induce hypergastrinemia, accelerate the onset of atrophic gastritis in H. pylori-positive patients (Klinkenberg-Knol, E. C. et al., 1994; Kuipers, E. J. et al., 1995; Kuipers, E. J. et al., 1996; Berstad, A. E. et al., 1997; Schenk, B. E., et al., 1998; Lamberts, R. et al., 2001). In general, the potential promotion of atrophic gastritis by PPIs may be due primarily to the decrease in gastric acid secretion, which leads to changes in the distribution of H. pylori and worsening of corpus gastritis. Indeed, acid suppression in mice has shown to produce similar changes in H. felis colonization within the murine stomach (Danon, S. J. et al., 1995). However, in most of the studies done primarily in GERD patients, there was a strong correlation between the degree of atrophic gastritis and serum gastrin levels. In one study, patients with the highest serum gastrin levels prior to PPI therapy showed the most marked progression in gastric atrophy during PPI therapy (Eissele, R. et al., 1997).

In order to explore the relative importance of achlorhydria versus hypergastrinemia in the pathogenesis of Helicobacter-mediated gastric atrophy, the effects of two acid suppressive reagents: the gastrin/CCK-B receptor antagonist YF47619 and the irreversible histamine H2 receptor antagonist loxtidine were examined (Colin-Jones, D. G. et al., 1995). Short-term studies utilizing the proton pump inhibitor omeprazole were examined for purposes of comparison. The three drugs act on different targets but all three inhibit gastric acid secretion and also induce hypergastrinemia. Importantly, the first two drugs inhibited progression of gastric preneoplasia while the third appeared to accelerate it. The results indicate the importance of the gastrin-histamine axis in the pathophysiology of Helicobacter-induced gastric atrophy and carcinogenesis, and may also have clinical implications for the development of safe approaches to chronic acid suppression.

### Example II

### YF476 and/or Loxtidine Treatment for 3 months Resulted in Synergistic Inhibition of Gastric Acid Output and Gastric Atrophy, Hyperplasia and Dysplasia in H. felis-infected INS-GAS Mice.

While the highly specific CCK-B/gastrin antagonist, YF476, and the irreversible H2 receptor blocker, loxtidine, have previously been shown to inhibit acid secretion in mice, they have not been studied in models of chronic Helicobacter infection. In addition, they have not previously been examined in a mouse model of gastric cancer such as the hypergastrinemic INS-GAS mouse model. Previous studies have demonstrated that young (<6 months old) INS-GAS mice have slightly elevated gastric acid secretion but that Helicobacter infection leads to a rapid reduction in gastric acid secretion (Cui, G. et al., 2003). Other groups have also shown an inhibitory effect of Helicobacter infection on gastric acid secretion (Dial, E. J. et al., 2000; Zhao C. M. et al., 2003). Consequently, the maximal gastric acid output (MAO) in using the pyloric ligation method in INS-GAS mice infected for 3 months with H. felis were examined. As shown in FIG. 7, YF476 or loxtidine treatment for 3 months (Group II or III) significantly inhibited MAO in H. felis-infected INS-GAS mice, and mice treated with both drugs (Group IV) exhibited essentially no acid output. The study also confirmed that 3-months H. felis infection resulted in a level of acid secretion that was lower than uninfected FVB/N control mice.

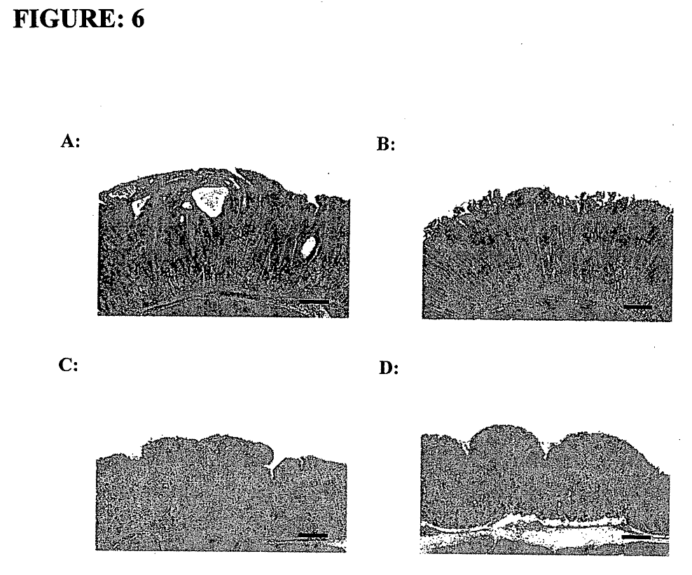

Helicobacter felis infection of INS-GAS mice also resulted in a rapid parietal cell loss and progression to gastric atrophy (Wang, T. C. et al., 2000). While YF476 and loxtidine are known acid inhibitors, an additional explanation for the profound inhibition of acid secretion observed in FIG. 7 may be that the drugs potentiated Helicobacter-mediated parietal cell loss. Thus, the gastric histology of the mice in each group were examined. As shown in Table 1 and FIG. 6, treatment with either YF476 alone or loxtidine alone (group II or III) appeared to result in slight inhibition of Helicobacter-mediated gastric atrophy. In addition, the prominent foveolar hyperplasia noted in the untreated INS-GAS mice was also partially reduced. In particular, the mice treated with the combination of YF476 and loxtidine showed almost complete inhibition of atrophy, gastric hyperplasia and dysplasia. Thus, YF476 and loxtidine appeared to have a synergistic inhibitory effect on both gastric acid output and in the development of gastric preneoplasia in H. felis-infected INS-GAS mice.

TABLE 1
Gastric mucosa histological scores in H. felis-infected INS-GAS
mice with YF476 and/or loxtidine treatment for 3 months.

| | | | | YF476 + |
|---|---|---|---|---|
| Corpus | No drug | YF476 | Loxtidine | Loxtidine |
| Inflammation | 1.88 ± 0.48 | 1.88 ± 0.25 | 1.83 ± 0.29 | 1.88 ± 0.63 |
| Atrophy | 2.13 ± 0.25 | 1.63 ± 0.75 | 1.83 ± 0.29 | 0.88 ± 0.48a |
| Hyperplasia | 2.25 ± 0.65 | 2.13 ± 0.63 | 1.33 ± 0.58 | 0.75 ± 0.29b |
| Dysplasia | 1.63 ± 0.63 | 1.38 ± 0.48 | 1.00 ± 0.50 | 0.38 ± 0.48b |

ap < 0.05; YF476 + loxtidine treated mice compared with no drug mice.
bp < 0.01; YF476 + loxtidine treated mice compared with no drug mice.
n = 4 for each group.

### Example III

### YF476 and Loxtidine Treatment for 6 months Resulted in Synergistic Inhibition of Gastric Carcinogenesis in H. felis-infected INS-GAS Mice.

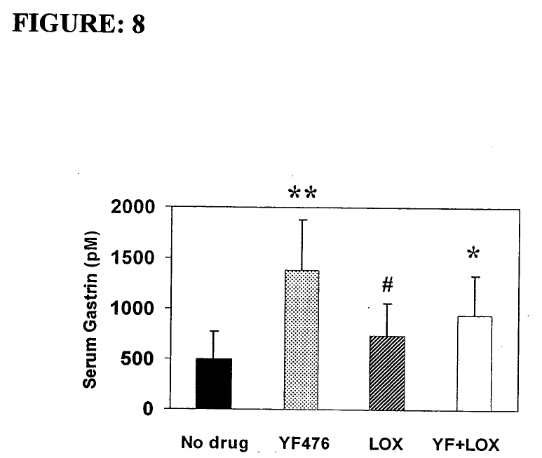

In order to determine the effect of CCK-B receptor and H-2 receptor blockade on the progression to gastric cancer, longer-term (6 month) drug treatment studies in our H. felis-infected INS-GAS mice were examined. Treatment with YF476 and loxtidine, through their inhibitory effects on acid production, have previously been shown to induce hypergastrinemia in non-transgenic rodents (Zhao, E. M. et al., 2003; Bjorkqvist, M. et al., 2002). Furthermore, H felis infection of the mildly hypergastrinemic INS-GAS mice has been shown to lead to worsening of the hypergastrinemia due primarily to progression of gastric atrophy (Wang, T. C. et al., 2000). Thus, not surprisingly, all four groups of H. felis-infected INS-GAS mice showed serum gastrin levels >500 pM. In the case of YF476 alone or YF476-loxtidine double-treated mice (group II and IV), the serum gastrin levels were significantly higher than those of the untreated mice, and in the mice treated with loxtidine alone (group III), there was a tendency toward higher gastrins that was not significant (p=0.054; FIG. 8). As previously reported in Wang et al., 2000, H.felis-infection of INS-GAS mice for more than 6 months resulted in high prevalence of gastric tumor in corpus area associated with marked gastric thickening. Consequently, the wet-tissue weights of stomachs from the mice treated with YF476 and/or loxtidine for 6 months were examined. Both the gross appearance (FIG. 5A-E) and the actual weights of stomachs from the mice treated with YF476 and/or loxtidine were much reduced compared with untreated H.felis-infected INS-GAS mice (FIG. 5F). In particular, the stomachs from mice treated withYF476 plus loxtidine (group IV) resembled closely those from uninfected non-transgenic normal FVB mice. The body weights of YF476 and/or loxtidine treated mice showed a significant increase from non-treated mice, suggesting overall improved health conditions (FIG. 4G). The H. felis-infected INS-GAS mice developed progressive cachexia after 6 months of infection, and this was largely ameliorated by treatment with YF476 and/or loxtidine. The ratios of stomach weight to body weight in the mice treated with YF476 and/or loxtidine were significantly lower than untreated infected INS-GAS mice. (FIG. 4H)

Histological examination and scoring of stomachs from YF476 and/or loxtidine treated mice confirmed an inhibitory effect by the drugs on progression to gastric neoplasia. As shown in FIG. 9B-C, treatment with either YF476 or loxtidine alone (group II or III) resulted in a significant decrease in overall mucosal thickness and the elimination of submucosal invasion observed in untreated mice, which resulted in a partial inhibition of progression to neoplasia (p<0.05 in Table 2; FIG. 9A-C). In addition, treatment with the combination of YF476 and loxtidine significantly improved histological scoring and resulted in nearly complete inhibition of neoplasia and normalization of histology with only mild inflammation and edema (p<0.01 in Table 2; FIG. 9A-C). Thus, YF476 and loxtidine appeared to have a synergistic inhibitory effect on gastric carcinogenesis in H. felis-infected INS-GAS mice.

TABLE 2
Gastric mucosa histological scores in H. felis-infected INS-GAS
mice with YF476 and/or loxtidine treatment for 6 months.

| | | | | YF476 + |
|---|---|---|---|---|
| Corpus | No drug | YF476 | Loxtidine | Loxtidine |
| Inflammation | 3.17 ± 0.26 | 2.50 ± 0.35b | 2.33 ± 1.17 | 2.25 ± 0.29b |
| Atrophy | 3.08 ± 0.20 | 3.10 ± 0.65 | 2.67 ± 0.61 | 2.25 ± 0.29b |
| Hyperplasia | 3.42 ± 0.20 | 3.10 ± 0.22a | 2.42 ± 1.07a | 1.63 ± 0.48b |
| Dysplasia | 3.25 ± 0.27 | 2.50 ± 0.61a | 2.17 ± 1.29a | 1.13 ± 0.25b |

ap < 0.05; YF476 and/or loxtidine treated mice compared with no drug mice.
bp < 0.01; YF476 and/or loxtidine treated mice compared with no drug mice.
n = 5 for each group.

### Example IV

### Treatment with YF476 or Loxtidine does not Reduce H. felis Colonization of Infected INS-GAS Mice

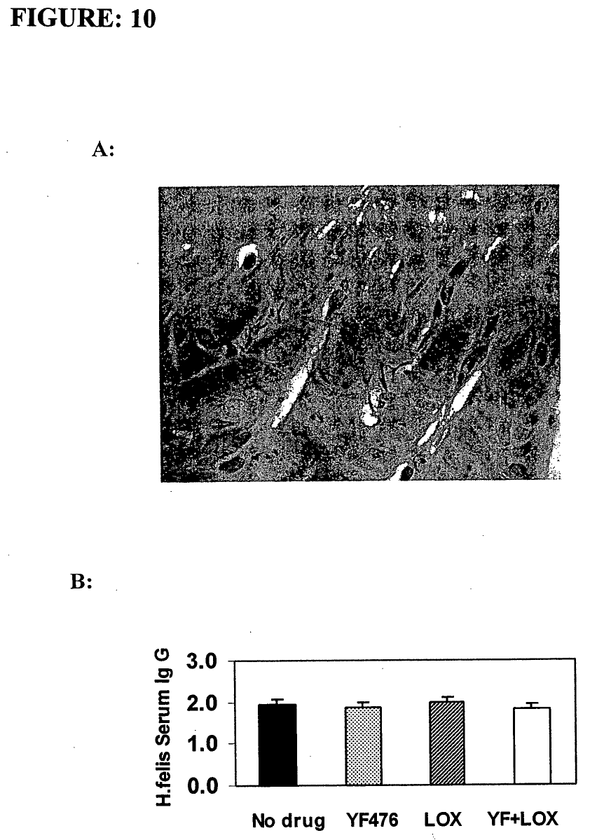

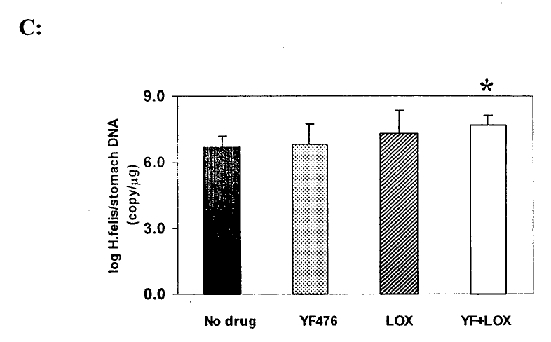

To elucidate the possible mechanisms for the synergistic inhibitory effect of YF476 and loxtidine on gastric atrophy and carcinogenesis in H. felis-infected INS-GAS mice, the possibility that drug treatment could block the ability of H. felis to colonize the stomachs of INS-GAS mice was examined. This question was addressed using three separate approaches. First, Warthin-Starry silver staining was performed to clarify whether H. felis organisms might in fact be eradicated by these acid suppressive reagents. These studies confirmed that H. felis could still be detected in the stomachs of YF476 and/or loxtidine-treated mice as non-treated mice, and that the shape of these bacteria appeared to be the spiral form, not coccoid in form. Secondly, ELISA assays of serum from the treated and untreated mice for H. felis-specific antibodies were performed. Results indicated a very similar IgG titer against H. felis among the four groups of mice (FIG. 10B). Lastly, DNA from mice gastric corpus for H. felis DNA using quantitative real-time PCR assay were analyzed. YF476 and/or loxtidine-treated mice showed a slight increase of H. felis DNA per stomach DNA, although this difference was not statistically significant (p>0.05 in FIG. 10C). In any case, these data indicate that the inhibitory effect of YF476 and loxtidine was not due to a significant reduction in H. felis colonization.

### Example V

### Growth Factor Expression Analysis in YF476 and/or Loxtidine-treated H. felis-infected INS-GAS Mice.

Gastrin appears to modulate growth and differentiation of the gastric mucosa both through direct as well as indirect actions (Dockray, G. J. et al., 2001). Two important classes of downstream growth factor targets that have been identified in recent years have included regenerating gene (Reg I) and the epidermal growth factor (EGF) family members such as heparin-binding EGF-like growth factor (HB-EGF), amphiregulin and transforming growth factor-alpha (TGF-alpha). Thus, to investigate the further possible mechanisms for the inhibitory effects of YF476 and loxtidine, the expression levels of various growth factors by quantitative realtime PCR were analyzed.

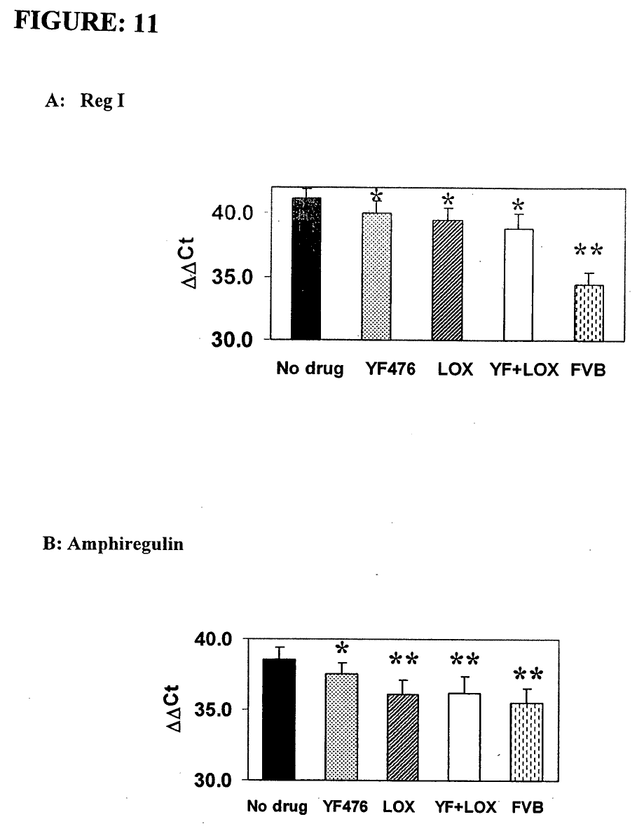

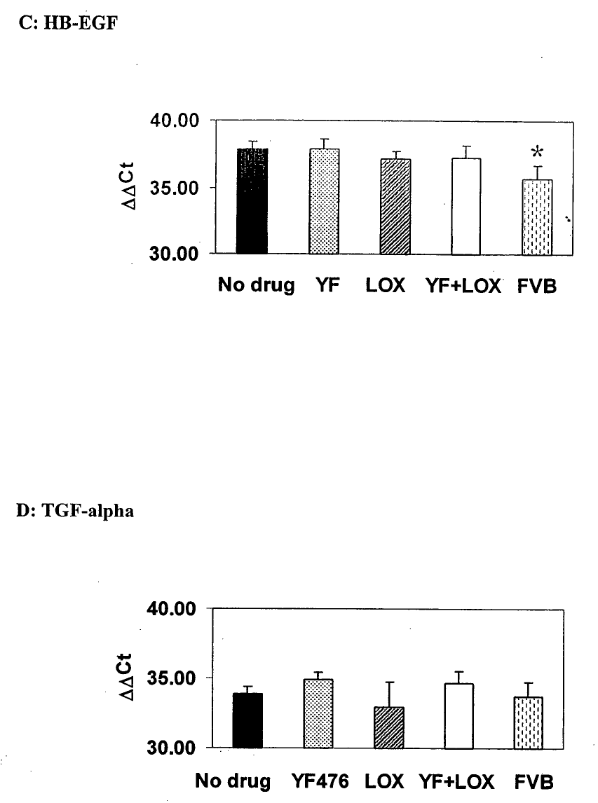

Among the four growth factors analyzed, the Reg I gene showed the highest ratio of upregulation in H. felis infected INS-GAS mice compared with wild type (FVB/N) mice. In response to treatment with YF476, loxtidine or both drugs, the level of expression was markedly reduced (FIG. 11A). Similarly, amphiregulin was also highly expressed in H. felis infected INS-GAS mice, and YF476 and/or loxtidine treatment significantly down-regulated the expression of this gene (FIG. 11B). In contrast, heparin binding EGF-like growth factor (HB-EGF) was mildly down-regulated by YF476 and/or loxtidine treatment, but this change was not statistically significant (FIG. 11C). In addition, another EGF family gene, transforming growth factor-alpha (TGF- alpha), did not show a significant change in response to YF476 and/or loxtidine treatment (FIG. 11D).

### Example VI

### Loxtidine Treatment of H. felis-infected INS-GAS Mice Resulted in a Mild Shift of Th1 to Th2 Polarization.

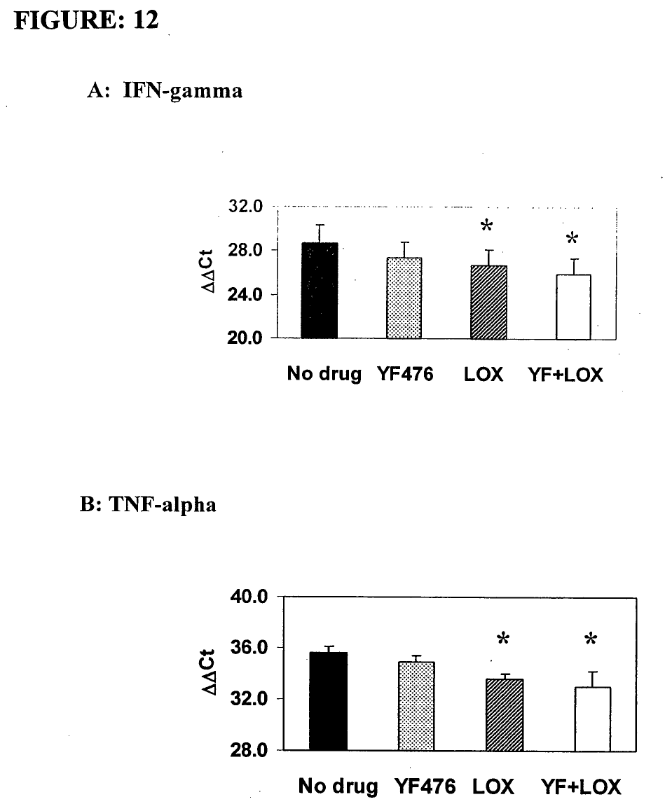

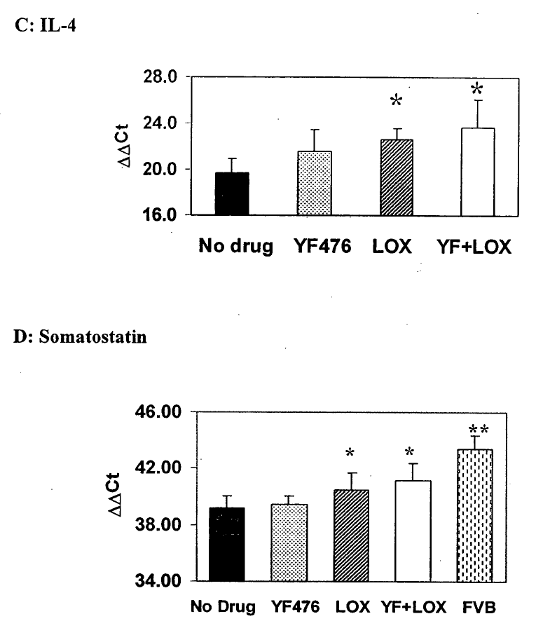

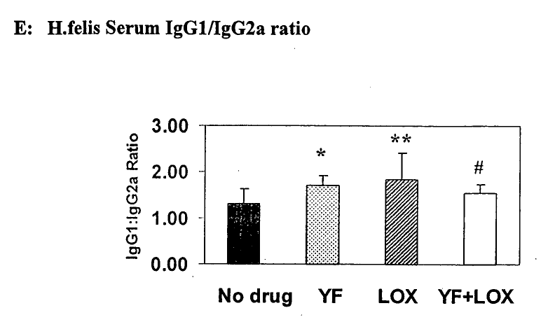

Given the importance of cytokine response in the pathogenesis of Helicobacter-induced gastric carcinogenesis, cytokine expression profiles by quantitative realtime PCR in the 4 groups of mice were analyzed. Th1 polarized cytokines such as interferon-gamma (IFN-γ) and tumor necrosis factor-alpha (TNF-γ) were significantly down-regulated in H.felis-infected INS-GAS mice treated with loxtidine alone or YF476 plus loxtidine (group III or IV) for 6 months (FIG. 12A,B). Treatment with YF476 alone (group II) resulted in a small but not statistically significant decrease in IFN-γ and TNF-alpha gene expression. In contrast, the cytokine IL-4 showed a slight but not statistically significant increase in YF476 treated mice, whereas large and significant increase in mice treated with loxtidine alone or YF476 plus loxtidine (FIG. 12C). In addition, given the previous report by Zavros, Y. et al., somatostatin expression in YF476 and/or loxtidine treated mice were analyzed (Zavros, Y. et al., 2003). As shown in FIG. 12D, loxtidine alone or in combination with YF476 showed significant down-regulation of somatostatin expression. These findings were confirmed with the investigation of serum immunoglobulin IgG1 versus IgG2a subclass titers for H.felis infection. As shown in FIG. 12E, YF476 or loxtidine treated mice (group II or III) showed significant up-regulation of the ratio of IgG 1 versus IgG2a titers, although the double treated mice (group IV) showed a slight increase in this ratio that was not significant. Taken together, loxtidine treatment, with or without YF476, resulted in a mild shift toward a Th2 polarized response to H. felis infection.

### Example VII

### Omeprazole Treatment for 3 months Resulted in Mild Progression of Gastric Hyperplasia and Dysplasia in H.felis-infected INS-GAS Mice.

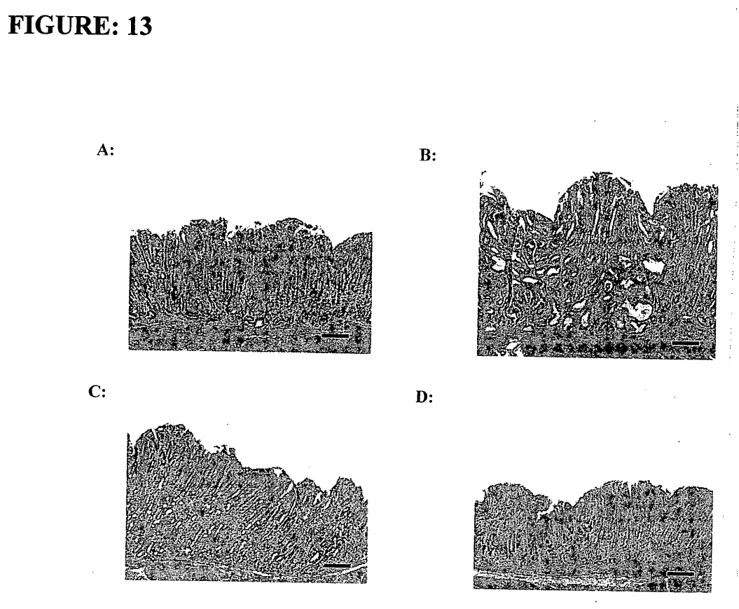

The effect of another type of acid suppressive reagent, the proton pump inhibitor omeprazole was also examined. In contrast to our findings with YF476 and/or loxtidine treatment, H. felis-infected INS-GAS mice treated with omeprazole for 3 months did not show a reduction in atrophy, but instead appeared to manifest a more rapid progression of gastric foveolar hyperplasia and dysplasia (Table 3; FIG. 13A,B). The combination of omeprazole with YF476 or loxtidine resulted in some improvement of histological features compared to treatment with omeprazole alone. In fact, treatment of H.felis-infected INS-GAS with the combination of omeprazole and YF476 led to significant suppression of gastric hyperplasia and dysplasia compared to omeprazole alone treated mice (Table 3; FIG. 13C). Furthermore, treatment with the combination of omeprazole and loxtidine resulted in remarkable suppression of gastric atrophy, hyperplasia and dysplasia (Table 3; FIG. 13D). In all treatment groups, the H. felis infection status did not change and serum gastrin levels were significantly higher than controls (data not shown).

TABLE 3
Gastric mucosa histological scores in H. felis-infected
INS-GAS mice with omeprazole alone or omeprazole with
YF476 or loxtidine treatment for 3 months

| | | | | Omeprazole + |
|---|---|---|---|---|
| Corpus | No drug | Omeprazole alone | Omeprazole + YF476 | Loxtidine |
| Inflammation | 0.88 ± 0.25 | 1.88 ± 0.63a | 1.00 ± 0.41b | 1.63 ± 0.48 |
| Atrophy | 3.13 ± 0.25 | 3.13 ± 0.25 | 2.88 ± 0.25 | 1.63 ± 0.25c |
| Hyperplasia | 1.88 ± 0.75 | 2.75 ± 0.50 | 1.00 ± 0.71c | 1.75 ± 0.29c |
| Dysplasia | 0.88 ± 0.25 | 1.38 ± 0.48 | 0.50 ± 0.71b | 0.75 ± 0.29b |

ap < 0.05; Omeprazole treated mice compared with no drug mice.
bp < 0.05; Omeprazole with YF476 or loxtidine treated mice compared with omeprazole alone treated mice.
cp < 0.01; Omeprazole with YF476 or loxtidine treated mice compared with Omeprazole alone treated mice.
n = 4 for each group.

Discussion of Examples II-VII

In this study, the effect of three distinct acid suppressive reagents: the gastrin/CCKB receptor antagonist YF476, the histamine H2 receptor antagonist loxtidine, and the proton pump inhibitor omeprazole were invenstigated on the H. felis-infected INS-GAS mouse model. All three drugs strongly inhibit gastric acid secretion and were initially developed as anti-ulcer or anti-GERD (gastro-esophageal reflux disease) drugs. Results strongly indicate that the gastrin-histamine axis contributes to the development of gastric atrophy and neoplasia. While YF476 and loxtidine both inhibited the atrophic and proliferative response in H. felis-infected hypergastrinemic mice, omeprazole appeared to worsen disease progression. Since all three regimens inhibit acid secretion and induce hypergastrinemia, the different responses seen with the three drugs support a possible pathogenic role for CCK-BR and H2R signaling in the INS-GAS/H. felis mouse model of gastric cancer.

The finding that YF476 could block disease progression was in keeping with the hypergastrinemic nature of the INS-GAS mouse model. YF476 is a potent and highly selective CCK-B/gastrin receptor antagonist that can also induce long-lasting suppression of acid secretion after a single injection. Previous in vivo studies have shown that it can inhibit many of the pathologic and proliferative effects induced by hypergastrinemia. Previous investigators have reported that the administration of YF476 to cotton rats for 6 months into cotton rats markedly reduced the development of spontaneous gastric carcinomas in this hypergastrinemic rodent model (Martinsen, T. C., et al., 2003). Current results provides further proof of the inhibitory effect of YF476 on the development of gastric carcinogenesis by showing that the gastric cancer observed in the INS-GAS mice is mediated at least in part by amidated gastrin.

The inhibitory effect of loxtidine was unexpected. In fact, the initial postulation was that treatment with an H2R antagonist would, through acid suppression and induction of hypergastrinemia, accelerate gastric carcinogenesis (Kobayashi, T. et al., 2000). Loxtidine is a well-studied irreversible and highly potent H2 receptor antagonist that has long been used as a model of hypergastrinemia. For example, it has been shown to induce gastric hyperplasia and ECL cell carcinoids in rodents (Fossmark, R. et al., 2000), similar to that observed with omeprazole (Viste, A. et al., 2004). Poynter et al., reported that loxtidine-treated rats showed ECL cell hyperplasia after 39 days administration, while similar lesions were observed after 28-days treatment with omeprazole (Poynter, D. et al., 1991). However, in contrast to effects of hypergastrinemia seen in the INS-GAS mouse model, long-term treatment of rats and mice with loxtidine did not result in loss of parietal cells, but if anything appeared to result in increased parietal cells (Brenna, E. et al., 1994). Similar observations have been noted in histamine deficient mice; histidine decarboxylase knockout (HDC-/-) mice kept on a low-histamine diet showed an expanded parietal cell pool despite exhibiting marked hypergastrinemia (Hunyady, B. et al., 2003). Thus, these earlier observations are consistent with current results, which suggest that loxtidine-treatment of H. felis-infected INS-GAS mice inhibits progression to atrophy. Taken together, these observations indicate that excessive histamine produced in response to gastrin stimulation may contribute to the gradual down-regulation of parietal cell number (gastric atrophy) and eventual progression to gastric cancer.

Current results indicated that inhibition of gastric disease progression occurred without significant change of H. felis colonization. Danon et al., reported that the localization of H. felis in the stomach was affected by gastric acid suppressive reagents, i.e., acid suppression caused the shift from antrum to corpus of H.felis colonies (Danon, S. J. et al., 1995). Thus, the observation of a slight (but not significant) increase of H. felis DNA would be consistent with this shift. Nevertheless, YF476 nor loxtidine treatment did not lead to a measurable change in H. felis infection status in overall, i.e., there was no decrease of serum H.felis-specific IgG titer nor no change in bacterial shape from spiral form to coccoid form. However, treatment with YF476 and/or loxtidine did result in significant down-regulation in the expression of Regenerating gene, Reg I. Previous reports have shown that the Reg I gene and protein, primarily expressed in gastric enterochromaffin-like (ECL) cells, are upregulated by gastrin (Fukui, H. et al., 1998; Fukui, H. et al., 2003) . In addition, transgenic overexpression of Reg I can result in a gastric phenotype that closely mirrors the hypergastrinemic phenotype (Murayama, Y. et al., 1995). Nevertheless, while Reg I is considered a gastrin-regulated gene, results with loxtidine indicate that the Reg I gene may also be regulated by histamine. Amphiregulin and heparin-binding EGF-like growth factor (HB-EGF) have been reported to be localized mainly in parietal cells of fundic glands, and their production by parietal cells was stimulated by gastrin and had a potent trophic effect for progenitor cells in the neck zone of the gastric fundic mucosa through EGF receptor (Murayama, Y. et al., 1995; Tsutsui, S. et al., 1997). In this study, amphiregulin was also significantly down-regulated in YF476 and/or loxtidine-treated mice, whereas expression of HB-EGF was not significantly changed in drug-treated mice. Another EGF family member, transforming growth factor-alpha (TGF-alpha), which has also been reported to modulate proliferation of the gastric mucosa, was not change in drug-treated mice.

The induction of gastric atrophy and neoplasia by Helicobacter-infection is strongly dependent on a strong Th1 immune response (Houghton, J. et al., 2002). In mice treated with loxtidine alone or YF476 plus loxtidine, Thl polarized cytokines such as IFN-γ or TNF-alpha were mildly down regulated in comparison with non-treated mice, whereas Th2 polarized cytokine such as IL-4 were mildly increased. Previous reports have indicated that shifting the immune response to gastric Helicobacter infection towards a Th2 polarized response resulted in significant protection from progression to atrophy and preneoplasia. Thus, concurrent enteric helminth infection in H. felis-infected mice leads to a substantial reduction in cytokines and chemokines associated with type 1 T-helper cells and reduced helicobacter-induced gastric atrophy (Fox, J. G. et al., 2003). Previous studies have documented important effects by histamine on the immune response. For example, studies employing H2R-deficient mice suggested that both Th1- and Th2-type responses are negatively regulated by H2R (Jutel, M. et al., 2001). However, other studies have recently shown that somatostatin is an important immune-modulatory factor, required for IL-4 upregulation in response to gastric inflammation (Zavros, Y. et al., 2003). Somatostatin was down-regulated in our hypergastrinemic mice, and as shown in FIG. 12C-D, loxtidine alone or the combination of YF476 and loxtidine inhibited substantially this down-regulation and promoted a Th2-polarized cytokine response. Thus, loxtidine may modulate inflammatory responses to Helicobacter infection both directly as well as indirectly through upregulation of somatostatin. Treatment with YF476 alone (group II mice) did not result in a significant change in the Th1/Th2 cytokine profile.

Studies in the past have investigated gastrin and histamine pathways in tumor development and progression. Histamine has been postulated to be an autocrine growth factor for some tumors. Histidine decarboxylase, the enzyme that produces histamine, is expressed in a number of cancers and tumor cell lines, and high concentrations of histamine can be detected in primary tumors such as colorectal (Boer, K. et al., 2003) and breast cancers (Garcia-Caballero, M. et al., 1994). Several clinical trials have showed positive benefits from treatment with H2 receptor antagonists such as cimetidine (Tonnesen, H., et al., 1998), although other studies have reported no benefit.

Gastrin has also been postulated to be a potential autocrine growth factor for cancer, although many of its effects in tumor growth may be through non-classical, non-CCK-B receptors (Dockray, G. J. et al., 2001). In addition to pharmacologic blockade of gastrin receptors, at least one group has attempted to use the gastrin immunogen gastrin-17-diphtheria toxoid (G17-DT; Gastrimmune) to induce anti-G17 antibodies for the treatment of gastric cancer (Watson, S. A. et al, 1999). Importantly, though, there have been no trials employing the combination of gastrin- and histamine-receptor antagonists. While murine studies described herein focus predominantly on the effects of the combination of CCK-BR and H2R blockade in prevention of Helicobacter-mediated carcinogenesis, the studies similar effects may be observed in cases of advanced cancer.

Current results also suggest that the combination of CCK-BR and H2R blockade may be useful as an approach to long-lasting acid suppression. The irreversible H2R blocker, loxtidine, was never developed as a drug for use in humans because of the association with gastric carcinoid and cancer in rats (Brittain, R. T. et al., 1985), despite the fact that a similar toxicity profile was found with proton pump inhibitors (Wetscher, G. J. et al., 1999; Viste, A. et al., 2004). Data accumulated from available histamine H2 receptor antagonists such as cimetidine, ranitidine and famotidine suggest that these agents when used alone showed no consistent effect on reducing the risk of gastric cancer development (Johnson, A. et al., 1996; Primrose, A. G. et al., 1998; Langman, M. J. et al,. 1999; La Vecchia, C. et al., 1002). The recently developed and highly specific CCK-B/gastrin receptor antagonists such as YF476 have not been clinically tested in human patients. Current findings show that the combination of these two drugs acts as powerful acid suppressant, as well as, in the prevention of Helicobacter-dependent carcinogenesis in a mouse model. Therefore, there is solid rationale for the development of this combination. Proton pump inhibitors are currently the most widely prescribed medication for acid suppression and treatment of GERD, and retrospective studies have consistently found PPIs to be effective over long (e.g. >11 years) periods of follow-up. Nevertheless, there are persistent concerns regarding the effects of chronic hypergastrinemia with respect to gastric atrophy, as well as possibly Barrett's esophagus (Haigh, C. R. et al., 2003). In the mouse model, omeprazole-treatment did result in acceleration in the development of gastric atrophy, consistent with earlier predictions and studies in GERD patients (Klinkenberg-Knol E. C. et al., 1994; Kuipers, E. J. et al., 1995; Kuipers, E. J. et al., 1996; Berstad, A. E. et al., 1997; Schenk, B. E. et al., 1998; Lamberts, R. et al., 2001). The treatment modalities described herein may be useful in developing strategies for long-term acid suppression.

The contents of all references, patents and published patent applications cited throughout this application, including the figures, are incorporated herein by reference.

Equivalents

Those skilled in the art will recognize, or be able to ascertain using no more than routine experimentation, many equivalents to the specific embodiments and methods described herein. Such equivalents are intended to be encompassed by the scope of the following claims.

References

Asahara, M., Y. Kinoshita, et al. (1994). “Gastrin receptor genes are expressed in gastric parietal and enterochromaffin-like cells of Mastomys natalensis.” Dig Dis Sci 39(10): 2149-56.

Berstad, A. E., J. G. Hatlebakk, et al. (1997). “Helicobacter pylori gastritis and epithelial cell proliferation in patients with reflux oesophagitis after treatment with lansoprazole.” Gut 41(6):740-7.

Bjorkqvist, M., C. Dornonville de la Cour, et al. (2002). “Role of gastrin in the development of gastric mucosa, ECL cells and A-like cells in newborn and young rats.” Regul Pept 108(2-3):73-82.

Boer, K., Z. Darvas, et al. (2003). “Expression of histidine decarboxylase in human colonic cancer cells and adenomatous polyps.” Inflamm Res 52 Suppl 1: S76-7.

Brenna, E., H. G. Swarts, et al. (1994). “Evaluation of the trophic effect of longterm treatment with the histamine H2 receptor antagonist loxtidine on rat oxyntic mucosa by differential counting of dispersed cells.” Gut 35(11):1547-50.

Brittain, R. T., D. Jack, et al. (1985). “Pharmacological basis for the induction of gastric carcinoid tumours in the rat by loxtidine, an insurmountable histamine H2-receptor blocking drug.” Br J Pharmacol 85(4):843-7.

Chen, D., C. M. Zhao, et al. (2004). “Altered control of gastric acid secretion in gastrin-cholecystokinin double mutant mice.” Gastroenterology 126(2):476-87.

Colin-Jones, D. G. (1995). “The role and limitations of H2-receptor antagonists in the treatment of gastro-oesophageal reflux disease.” Aliment Pharmacol Ther 9 Suppl 1:9-14.

Cui G et al. (2003) “Overexpression of glycine-extended gastrin maintains acid secretion and prevents the development of preneoplasia in the stomach via reducing parietal loss in transgenic mice” Gastroenterology 124 (4) suppl.: A-141

Danon, S. J., J. L. O'Rourke, et al. (1995). “The importance of local acid production in the distribution of Helicobacter felis in the mouse stomach.” Gastroenterology 108(5): 1386-95.

Dial, E. J., L. R. Hall, et al. (2000). “Altered gastrin regulation in mice infected with Helicobacter felis.” Dig Dis Sci 45(7):1308-14.

Dockray, G. J., A. Varro, et al. (2001). “The gastrins: their production and biological activities.” Annu Rev Physiol 63:119-39.

Eissele, R., G. Brunner, et al. (1997). “Gastric mucosa during treatment with lansoprazole: Helicobacter pylori is a risk factor for argyrophil cell hyperplasia.” Gastroenterology 112(3):707-17.

Fossmark, R., T. C. Martinsen, et al. (2004). “ECL-cell derived gastric cancer in male cotton rats dosed with the H2-blocker loxtidine.” Cancer Res 64(10):3687-93.

Fox, J. G., P. Beck, et al. (2000). “Concurrent enteric helminth infection modulates inflammation and gastric immune responses and reduces helicobacter-induced gastric atrophy.” Nat Med 6(5):536-42.

Fox, J. G., A. B. Rogers, et al. (2003). “Helicobacter pylori-associated gastric cancer in INS-GAS mice is gender specific.” Cancer Res 63(5):942-50.

Fox, J. G., T. C. Wang, et al. (2003). “Host and microbial constituents influence Helicobacter pylori-induced cancer in a murine model of hypergastrinemia.” Gastroenterology 124(7):1879-90.

Fukui, H., Y. Kinoshita, et al. (1998). “Regenerating gene protein may mediate gastric mucosal proliferation induced by hypergastrinemia in rats.” Gastroenterology 115(6): 1483-93.

Fukui, H., F. Franceschi, et al. (2003). “Effects of Helicobacter pylori infection on the link between regenerating gene expression and serum gastrin levels in Mongolian gerbils.” Lab Invest 83(12):1777-86.

Garcia-Caballero, M., E. Neugebauer, et al. (1994). “Histamine synthesis and content in benign and malignant breast tumours. Its effects on other host tissues.” Surg Oncol 3(3): 167-73.

Ge, Z., D. A. White, et al. (2001). “Fluorogenic PCR-based quantitative detection of a murine pathogen, Helicobacter hepaticus.” J Clin Microbiol 39(7):2598-602.

Haigh, C. R., S. E. Attwood, et al. (2003). “Gastrin induces proliferation in Barrett's metaplasia through activation of the CCK2 receptor.” Gastroenterology 124(3):615-25.

Helicobacter and Cancer Collaborative Group (2001) “Gastric cancer and Helicobacter pylori: a combined analysis of 12 case control studies nested within prospective cohorts.” Gut 49(3):347-53.

Houghton, J., J. G. Fox, et al. (2002). “Gastric cancer: laboratory bench to clinic.” J Gastroenterol Hepatol 17(4):495-502.

Hunyady, B., A. Zolyomi, et al. (2003). “Expanded parietal cell pool in transgenic mice unable to synthesize histamine.” Scand J Gastroenterol 38(2):133-40.

International Agency for Research on Cancer (1994) “Infection with Helicobacter pylori.” IARC monographs on the evaluation of the carcinogenic risks to humans. Vol. 61. p177.

Johnson, A. G., S. S. Jick, et al. (1996). “Histamine-2 receptor antagonists and gastric cancer.” Epidemiology 7(4):434-6.

Jutel, M., T. Watanabe, et al. (2001). “Histamine regulates T-cell and antibody responses by differential expression of H1 and H2 receptors.” Nature 413(6854):420-5.

Kazumori, H., S. Ishihara, et al. (2001). “Analysis of gastrin receptor gene expression in proliferating cells in the neck zone of gastric fundic glands using laser capture microdissection.” FEBS Lett 489(2-3):208-14.

Kidd, M., L. H. Tang, et al. (2000). “Gastrin-mediated alterations in gastric epithelial apoptosis and proliferation in a mastomys rodent model of gastric neoplasia.” Digestion 62(2-3):143-51.

Klinkenberg-Knol, E. C., H. P. Festen, et al. (1994). “Long-term treatment with omeprazole for refractory reflux esophagitis: efficacy and safety.” Ann Intern Med 121(3):161-7.

Kobayashi, T., et al. 2000. Abnormal functional and morphological regulation of the gastric mucosa in histamine H2 receptor-deficient mice. J. Clin. Invest. 105:1741-1749

Kuipers, E. J., A. M. Uyterlinde, et al. (1995). “Increase of Helicobacter pylori-associated corpus gastritis during acid suppressive therapy: implications for long-term safety.” Am J Gastroenterol 90(9):1401-6.

Kuipers, E. J., L. Lundell, et al. (1996). “Atrophic gastritis and Helicobacter pylori infection in patients with reflux esophagitis treated with omeprazole or fundoplication.” N Engl J Med 334(16):1018-22.

La Vecchia, C. and A. Tavani (2002). “A review of epidemiological studies on cancer in relation to the use of anti-ulcer drugs.” Eur J Cancer Prev 11(2):117-23.

Langman, M. J., J. A. Dunn, et al. (1999). “Prospective, double-blind, placebo-controlled randomized trial of cimetidine in gastric cancer. British Stomach Cancer Group.” Br J Cancer 81(8):1356-62.

Lamberts, R., G. Brunner, et al. (2001). “Effects of very long (up to 10 years) proton pump blockade on human gastric mucosa.” Digestion 64(4):205-13.

Livak, K. J. and T. D. Schmittgen (2001). “Analysis of relative gene expression data using real-time quantitative PCR and the 2(-Delta Delta C(T)) Method.” Methods 25(4): 402-8.

Martinsen, T. C., S. Kawase, et al. (2003). “Spontaneous ECL cell carcinomas in cotton rats—natural course and the prevention by a gastrin receptor antagonist.” Carcinogenesis 29:29.

Miyaoka Y et al, (2004) “Transgenic overexpression of Reg protein caused gastric cell proliferation and differentiation along parietal cell and chief cell lineages. Oncogene 23(20):3572-9.

Murayama, Y., J. Miyagawa, et al. (1995). “Localization of heparin-binding epidermal growth factor-like growth factor in human gastric mucosa.” Gastroenterology 109(4): 1051-9.

Poynter, D. and S. A. Selway (1991). “Neuroendocrine cell hyperplasia and neuroendocrine carcinoma of the rodent fundic stomach.” Mutat Res 248(2):303-19.

Primrose, J. N., G. V. Miller, et al. (1998). “A prospective randomised controlled study of the use of ranitidine in patients with gastric cancer. Yorkshire GI Tumour Group.” Gut 42(1):17-9.

Schenk, B. E., E. J. Kuipers, et al. (1998). “Hypergastrinaemia during long-term omeprazole therapy: influences of vagal nerve function, gastric emptying and Helicobacter pylori infection.” Aliment Pharmacol Ther 12(7):605-12.

Takinami, Y., H. Yuki, et al. (1997). “YF476 is a new potent and selective gastrin/cholecystokinin-B receptor antagonist in vitro and in vivo.” Aliment Pharmacol Ther 11(1):113-20.

Tang, L. H., E. A. Luque, et al. (1997). “Gastrin receptor expression and function during rapid transformation of the enterochromaffin-like cells in an African rodent.” Regul Pept 72(1):9-18.

Tonnesen, H., U. Knigge, et al. (1988). “Effect of cimetidine on survival after gastric cancer.” Lancet 2(8618):990-2.

Tsutsui, S., Y. Shinomura, et al. (1997). “Induction of heparin binding epidermal growth factor-like growth factor and amphiregulin mRNAs by gastrin in the rat stomach.” Biochem Biophys Res Commun 235(3):520-3.

Uemura, N., S. Okamoto, et al. (2001). “Helicobacter pylori infection and the development of gastric cancer.” N Engl J Med 345(11):784-9.

Viste, A., K. Ovrebo, et al. (2004). “Lanzoprazole promotes gastric carcinogenesis in rats with duodenogastric reflux.” Gastric Cancer 7(1):31-5.

Wang, T C. et al. (2000). “Synergistic Interaction Between Hypergastrinemia and Helicobacter Infection in a Mouse Model of Gastric Cancer.” Gastroenterology 118:36-47

Wang, T. C., S. Bonner-Weir, et al. (1993). “Pancreatic gastrin stimulates islet differentiation of transforming growth factor alpha-induced ductular precursor cells.” J Clin Invest 92(3):1349-56.

Watson, S. A., T. M. Morris, et al. (1999). “A comparison of the therapeutic effectiveness of gastrin neutralisation in two human gastric cancer models: relation to endocrine and autocrine/paracrine gastrin mediated growth.” Gut 45(6):812-7.

Wetscher, G. J., R. A. Hinder, et al. (1999). “Gastric acid blockade with omeprazole promotes gastric carcinogenesis induced by duodenogastric reflux.” Dig Dis Sci 44(6): 1132-5.

Zavros, Y., S. Rathinavelu, et al. (2003). “Treatment of Helicobacter gastritis with IL-4 requires somatostatin.” Proc Natl Acad Sci U S A 100(22):12944-9.

Zhao, C. M., X. Wang, et al. (2003). “Chronic Helicobacter pylori infection results in gastric hypoacidity and hypergastrinemia in wild-type mice but vagally induced hypersecretion in gastrin-deficient mice.” Regul Pept 115(3):161-70.

1. A method for treatment of a subject having acid peptic disorders, comprising administering a therapeutically effective amount of a histamine 2-receptor antagonist and a gastrin receptor antagonist, such that the subject is treated.

2. The method according to claim 1, wherein the acid peptic disorders is selected from the group comprising of acid reflux disease (GERD), peptic ulcer disease, dyspepsia, gastritis, and pre-malignant and malignant disease of the stomach.

3. A method for treatment of a subject having proliferative disorders, comprising administering a therapeutically effective amount of a histamine 2-receptor antagonist and a gastrin receptor antagonist, such that the subject is treated.

4. The method according to claim 3, wherein the proliferative disorder is cancer.

5. The method according to claim 4, wherein the cancer is selected from the group comprising of colon, ovarian, lung, breast, endometrial, uterine, hepatic, gastrointestinal, prostate, and brain cancer; tumorigenesis and metastasis; skeletal dysplasia; and hematopoietic and myeloproliferative disorders.

6. The method according to claim 1, wherein the histamine 2-receptor antagonist is a reversible inhibitor.

7. The method according to claim 6, wherein the reversible inhibitor is selected from the group comprising of nizatidine, cimetideine, ranitidine, famotidine and roxatide.

8. The method according to claim 1, wherein the histamine 2-receptor antagonist is a irreversible inhibitor.

9. The method according to claim 8, wherein the irreversible inhibitor is selected from the group comprising of loxtidine and lamitidine.

10. The method according to claim 9, wherein the irreversible inhibitor is loxtidine.

11. The method of any one of the preceding claims, wherein the gastrin receptor antagonist is selected from the group consisting of YF476, YM022 and JB93182.

12. The method of any one of the preceding claims, wherein the therapeutically effective amount comprises from about 0.02 to about 0.5 mg/kg/day of the histamine 2-receptor antagonist.

13. The method of any one of the preceding claims, wherein the therapeutically effective amount comprises from about 1 to 25 mg/kg/day of the gastrin receptor antagonist.

14. The method of any one of the preceding claims, wherein the treatment results in a diminution in gastric acid secretion.

15. A method of any one of the preceding claims, wherein the histamine 2-receptor antagonist and the gastrin receptor antagonist are simultaneously administered to a subject.

16. A method of any one of the preceding claims, wherein the histamine 2-receptor antagonist and the gastrin receptor antagonist when simultaneously present in a subject act synergistically to reduce, inhibit, or ameliorate the symptoms or pathogenesis of acid peptic disorders.

17. A pharmaceutical composition comprising a therapeutically effective amount of a histamine 2-receptor antagonist and a gastrin receptor antagonist.

18. The pharmaceutical composition of claim 17, wherein the composition is formulated for oral administration.

19. The pharmaceutical composition of claim 18, wherein the composition is a capsule.

20. The capsule of claim 19, wherein the capsule is selected from the group consisting of a starch capsule, a hard gelatin capsule and a soft gelatin capsule.

21. The pharmaceutical composition according to claim 17, wherein the Histamine 2-receptor antagonist and the gastrin receptor antagonist are packaged in separate containers for sale or delivery to consumers.


SEQUENCE LISTING (18 sequences)

SEQ ID NO: 1 - DNA, 24 bp, Artificial Sequence
  Primer
ttcgattggtcctacaggctcaga

SEQ ID NO: 2 - DNA, 27 bp, Artificial Sequence
  Primer
ttcttgttgatgacattgaccaacgca

SEQ ID NO: 3 - DNA, 22 bp, Artificial Sequence
  Primer
aaggagagtggcactacagacg

SEQ ID NO: 4 - DNA, 22 bp, Artificial Sequence
  Primer
gtattgggcatcacagttgtca

SEQ ID NO: 5 - DNA, 22 bp, Artificial Sequence
  Primer
gacccatgcctcaggaaataca

SEQ ID NO: 6 - DNA, 22 bp, Artificial Sequence
  Primer
tacagccaccacagccaagact

SEQ ID NO: 7 - DNA, 22 bp, Artificial Sequence
  Primer
ggcaaaaatggaaaaggcagaa

SEQ ID NO: 8 - DNA, 22 bp, Artificial Sequence
  Primer
cgaggatgatggcagagacaaa

SEQ ID NO: 9 - DNA, 22 bp, Artificial Sequence
  Primer
gccggtttttggtgcaggaaga

SEQ ID NO: 10 - DNA, 22 bp, Artificial Sequence
  Primer
ttgcggagctgacagcagtgga

SEQ ID NO: 11 - DNA, 24 bp, Artificial Sequence
  Primer
catggctgtttctggctgttactg

SEQ ID NO: 12 - DNA, 24 bp, Artificial Sequence
  Primer
gttgctgatggcctgattgtcttt

SEQ ID NO: 13 - DNA, 22 bp, Artificial Sequence
  Primer
tggcccagaccctcacactcag

SEQ ID NO: 14 - DNA, 20 bp, Artificial Sequence
  Primer
acccatcggctggcaccact

SEQ ID NO: 15 - DNA, 22 bp, Artificial Sequence
  Primer
atcggcattttgaacgaggtca

SEQ ID NO: 16 - DNA, 18 bp, Artificial Sequence
  Primer
catcgaaaagcccgaaag

SEQ ID NO: 17 - DNA, 22 bp, Artificial Sequence
  Primer
gtcctggctttgggcggtgtca

SEQ ID NO: 18 - DNA, 22 bp, Artificial Sequence
  Primer
tgcagctccagcctcatctcgt